(19) United States
(12) Patent Application Publication
(10) Pub. No.: US 2026/0002174 A1
(43) Pub. Date: Jan. 1, 2026

(54) GENE THERAPIES FOR TREATMENT OF INFANTILE NEUROAXONAL DYSTROPHY

(71) Applicant: INADcure Foundation Inc., Fairfield, NJ (US)

(72) Inventors: Neil Hackett; Amanda Hope; Leena Panwala

(21) Appl. No.: 18/866,921

(22) PCT Filed: May 18, 2023
(86) PCT No.: PCT/US2023/067167

§ 371 Date: Nov. 18, 2024

Related U.S. Application Data
(60) Provisional application No. 63/343,379, filed on May 18, 2022.
(60) Provisional application No. 63/384,014, filed on Nov. 16, 2022.

(51) Int. Cl. C12N 15/86; A61K 38/46; A61K 48/00; A61P 25/00; C07K 14/005; C12N 9/18

(52) U.S. Cl. CPC C12N 15/86; A61K 38/465; A61K 48/005; A61P 25/00; C07K 14/005; C12N 9/18; C12Y 301/01004; C12N 2750/14122; C12N 2750/14143; C12N 2830/50

(57) ABSTRACT

The present invention provides gene therapies for the treatment of Infantile Neuroaxonal Dystrophy. The invention provides a viral vector comprising a viral capsid and an expression cassette comprising a nucleic acid encoding the PLA2G6 gene. The expression cassette comprises, in order, a SYN1 promoter or an EF1a promoter, a nucleic acid sequence encoding a PLA2G6 gene, and a poly(A) signal. Advantageously, the expression cassette does not comprise a woodchuck hepatitis virus post-transcriptional regulatory element (WPRE) between the nucleic acid sequence encoding a PLA2G6 gene and the poly(A) signal.

### FIELD OF THE INVENTION

The present invention relates to gene therapies for the treatment of infantile neuroaxonal dystrophy (INAD).

### SEQUENCE LISTING

This application contains a sequence listing which has been submitted in extensible Markup Language (XML) format via the Patent Center and is hereby incorporated by reference in its entirety. The XML-formatted sequence listing, created on May 18, 2023, is named INAD-001-01WO-ST26, and is 8,532 bytes in size.

### BACKGROUND

Infantile Neuroaxonal Dystrophy (INAD) is an extremely rare developmental disorder that primarily affects the nervous system. In 2022, there were only about 150 cases known globally. Individuals with infantile neuroaxonal dystrophy typically do not have any symptoms at birth, but at approximately 18 months begin to experience delays in acquiring new motor and intellectual skills, such as crawling or beginning to speak. Seizures may present at any time of disease progression. The progression is usually rapid, and patients rarely survive beyond their first decade, even with supportive care.

Mutations in the PLA2G6 gene have been identified in most individuals with infantile neuroaxonal dystrophy. The PLA2G6 gene provides instructions for making an enzyme called an A2 phospholipase, involved in metabolizing phospholipids. Different mutations in the PLA2G6 gene cause clinically distinct phenotypes. As a result, each case presents many different known and novel causative mutations and differing clinical presentation and progression rates.

Unfortunately, given the limited and clinically diverse patient population, investment in the development of potential therapies for INAD has been limited. As a consequence, only palliative treatments are available for children suffering from INAD and the life expectancy for these children is just 5 to 10 years.

### SUMMARY OF THE INVENTION

The present invention provides gene therapies for the treatment of Infantile Neuroaxonal Dystrophy. Gene therapies of the invention provide a wildtype copy of the PLA2G6 gene to the correct cell types at the correct level. By providing an additional wildtype copy of the PLA2G6 gene, A2 phospholipase expression can be restored, directly treating the cause of INAD.

Aspects of the invention provide a viral vector comprising a viral capsid and an expression cassette comprising a nucleic acid encoding the PLA2G6 gene. The expression cassette comprises, in order, a SYN1 promoter or an EF1a promoter, a nucleic acid sequence encoding a PLA2G6 gene, and a poly(A) signal.

Advantageously, the expression cassette does not comprise a woodchuck hepatitis virus post-transcriptional regulatory element (WPRE) between the nucleic acid sequence encoding a PLA2G6 gene and the poly(A) signal. For example, the entirety of the expression cassette may not comprise a woodchuck hepatitis virus post-transcriptional regulatory element. This is advantageous, because although WPRE may increase mRNA stability and enhances transgene expression. WPRE is not advisable for clinical use due to its oncogenic potential (Kingsman, Mitrophanous et al. 2005). The present invention, for the first time, shows that a vector, which lacks this element, will express PLA2G6 at a clinically sufficient level.

The viral capsid may be an AAV9 capsid. The vector may comprise inverted terminal repeats (ITRs) flanking the expression cassette. The ITRs may be AAV2 ITRs.

The vector may enable expression of the expression cassette in all tissue types. For example, the viral vector may enable expression of the expression cassette non-neuronal tissue. The vector may enable expression of the expression cassette in muscular tissue.

Without being bound to a mechanism of action, expression of the cassette in all tissue types may be facilitated by the promoter in the cassette. For example, the vector may comprise a SYN1 promoter or the vector may comprise an EF1a promoter.

The nucleic acid sequence encoding the PLA2G6 gene may be SEQ ID NO. 1. SEQ ID NO: 1 is the membrane bound isoform 1 of the PLA2G6 gene. The nucleic acid sequence may also encode any isoform of PLA2G6. For example, the nucleic acid sequence may encode isoform 2 of the PLA2G6 gene, the cytoplasmic isoform of the gene. The sequence provides a corrected sequence of the PLA2G6 gene, correcting the genetic mutations in the gene causing INAD.

Accordingly, aspects of the invention provide a method of treating Infantile Neuroaxonal Dystrophy in a subject. The method comprises administering to the subject a composition comprising a viral capsid and an expression cassette comprising a nucleic acid encoding the PLA2G6 gene. The expression cassette comprises, in order, a SYN1 and/or EF1a promoter; a nucleic acid sequence encoding a PLA2G6 gene, and a poly(A) signal.

Advantageously, the expression cassette does not comprise a woodchuck hepatitis virus post-transcriptional regulatory element (WPRE) between the nucleic acid sequence encoding a PLA2G6 gene and the poly(A) signal. The entirety of the expression cassette may not comprise a woodchuck hepatitis virus post-transcriptional regulatory element.

In methods of the invention, the viral capsid may be an AAV9 capsid. The vector may comprise inverted terminal repeats (ITRs) flanking the expression cassette. The ITRs may be AAV2 ITRs. The viral vector may enable expression of the expression cassette non-neuronal tissue. The vector may enable expression of the expression cassette in muscular tissue. The vector may comprise a SYN1 promoter. The vector may comprise an EF1a promoter.

The nucleic acid sequence encoding the PLA2G6 gene may encode any isoform of PLA2G6.

Aspects of the invention further provide a gene therapy in which both isoforms of the PLA2G6 gene are expressed from the AAV vector simultaneously. The nucleic acid encoding the PLA2G6 gene may encode both isoforms of the PLA2G6 gene. The nucleic acid encoding the PLA2G6 gene may comprise a hybrid of cDNA and genomic DNA. The nucleic acid may allow for alternate splicing that results in a mixture of mRNAs with and without exon 9. The nucleic acid may comprise cDNA for exons 2-8 of the PLA2G6 gene. The nucleic acid may comprise genomic DNA configurations for intron 8, exon 9, and intron 9. The nucleic acid may comprise cDNA for exons 9-17. The nucleic acid sequence may not comprise exon 1 of the PLA2G6 gene.

The nucleic acid may comprise forms of introns 8 and 9 of the PLA2G6 gene in which portions of the center of introns 8 and 9 are removed without removing the ends of introns 8 and 9 of the PLA2G6 gene. The nucleic acid may comprise SEQ ID NO: 2.

Advantageously, the novel nucleic acid constructs of the invention described above may be packaged into an AAV vector.

### BRIEF DESCRIPTION OF THE DRAWINGS

FIG. 1 is a graph of the general age-dependent decrease in the INAD rating scale score.

FIG. 2 is a schematic of an expression cassette (AAV9- -hPLA2G6) of the invention.

FIG. 3 is Table 3, a summary of pre-clinical efficacy studies.

FIG. 4 is a graph of the impact of AAV9-hPLA2G6 (UCL) on survival in a missense mouse model of INAD.

FIG. 5 is a graph of the impact of AAV9-hPLA2G6 (UCL) on rotarod latency in missense mouse model of INAD

FIG. 6 is a graph of the impact of AAV9-hPLA2G6 (UCL) on neuron density.

FIG. 7 is an image of the impact of AAV9-hPLA2G6 (UCL) on lysosomal function in neurons.

FIG. 8 is a graph of the impact of AAV9-hPLA2G6 (UCL) on lysosomal function in neurons.

FIG. 9 is an image of the impact of AAV9-hPLA2G6 (UCL) on astrocyte activation.

FIG. 10 is a graph of the impact of AAV9-hPLA2G6 (UCL) on astrocyte activation.

FIG. 11 is a graph of impact of AAV9-hPLA2G6 on rotarod latency in missense mouse model of INAD.

FIG. 12 is Table 9, a simplified schedule of short-term safety assessments.

FIG. 13 is Table 10, a simplified schedule of long-term efficacy assessments.

FIG. 14 is a schematic of a cDNA/genomic hybrid nucleic acid of the invention encoding the of the PLA2G6 gene.

FIG. 15 is a schematic of a cDNA/genomic hybrid nucleic acid of the invention encoding the of the PLA2G6 gene.

### DETAILED DESCRIPTION

The present invention provides gene therapies for the treatment of Infantile Neuroaxonal Dystrophy. The invention provides a viral vector comprising a viral capsid and an expression cassette comprising a nucleic acid encoding the PLA2G6 gene. The expression cassette comprises, in order, a SYN1 promoter or an EF1a promoter, a nucleic acid sequence encoding a PLA2G6 gene, and a poly(A) signal. Advantageously, the expression cassette does not comprise a woodchuck hepatitis virus post-transcriptional regulatory element (WPRE) between the nucleic acid sequence encoding a PLA2G6 gene and the poly(A) signal.

### Infantile Neuroaxonal Dystrophy (INAD)

Infantile neuroaxonal dystrophy (INAD) is an autosomal recessive genetic disorder resulting from mutations in the phospholipase A2 type VI protein (PLA2G6) gene, which cause significantly decreased or absent phospholipase activity.

INAD is a subset of Phospholipase A2 group VI (PLA2G6)-associated neurodegeneration (PLAN) which encompasses a set of neurodegenerative disorders with heterogeneous clinical presentation but with a common genetic origin (Iodice, Spagnoli et al. 2017). In one classification, there are four categories dependent on the age of onset:
infantile neuroaxonal dystrophy (INAD)
atypical neuroaxonal dystrophy (ANAD)
parkinsonian syndrome, which contains adult-onset dystonia-parkinsonism (DP)
autosomal recessive early-onset parkinsonism (AREP)

Therefore, different mutations in the same gene cause clinically distinct phenotypes. This has been investigated by assessing the in vitro catalytic function of the respective mutant PLA2G6 enzymes, (Engel, Jing et al. 2010). Mutations associated with INAD caused loss of enzyme activity, with mutant proteins exhibiting less than 20% of the specific activity of wild-type protein with both lysophospholipids and phospholipids as substrate. In contrast, mutations associated with dystonia-parkinsonism do not impair catalytic activity. Two of the mutations produce a significant increase in specific activity for phospholipid but not lysophospholipid substrates. This proposal is exclusively concerned with INAD, but the data generated may be subsequently helpful in developing treatments for all forms of PLAN.

The onset of INAD typically is observed at ˜18 months of age with developmental delay or early regression often presenting as loss of balance, (Gregory and Hayflick 2005, Meyer, Kurian et al. 2015, Hayflick, Kurian et al. 2018). Examination reveals gross and fine motor regression, gait disturbance, truncal hypotonia, and in some patients, strabismus, and nystagmus. The disease progresses into spastic tetraparesis with symmetric pyramidal signs, progressive cognitive decline, optic atrophy, and bulbar dysfunction. Seizures may present at any time of disease progression in ˜43% of patients. The progression is usually rapid, and patients rarely survive beyond their first decade, even with supportive care. Histopathology may show axonal spheroid bodies in both the central and peripheral nervous system, and neuroimaging usually reveals cerebellar atrophy and, in some cases, iron accumulation in the globus pallidus.

Multiple lines of evidence suggest that neuronal loss is a critical aspect of disease progression. In peripheral nerve biopsies and post-mortem material, the accumulation of spheroid bodies in the nerves is observed. In one study, eight subjects with PLAN were assessed by clinical, radiological, electroencephalographic (EEG), and electrodiagnostic testing (Gitiaux, Kaminska et al. 2018). All patients presented marked motor axonal loss, with decreased or absent distal compound muscle action potentials, acute and chronic denervation at needle electromyography, in contrast with preservation of sensory conduction. In MRI imaging studies, there is a rapidly progressing cerebellar atrophy. Some MRIs also show thin optic chiasma, signal hyperintensity of the dentate nuclei and white matter, and cerebral cortical atrophy (Farina, Nardocci et al. 1999, Mascalchi, Mari et al. 2017).

INAD is an extremely rare disease, with only 150 cases known globally. Each case presents many different known and novel causative mutations and differing clinical presentation and progression rates.

Clinical trials of potential therapies for INAD benefit from the INAD rating scale, (Atwal, Midei et al. 2020). This is a validated scale involving a structured neurological examination for INAD (scored out of 80). The study includes six main categories of pediatric developmental evaluation: 1) gross motor-and-truncal-stability skills, 2) fine motor skills, 3) bulbar function, 4) ocular function, 5) temporal-frontal function, and 6) functional evaluation of the autonomic nervous system. A cohort of patients diagnosed with INAD was followed prospectively to validate the score against disease severity and progression. There was a significant correlation between the total neurological assessment score and months since symptom onset, with a highly statistically significant correlation between assessment score and disease onset. As hypothesized, the coefficient of months-since-symptom-onset is strongly negative, indicating a negative correlation between total score and months since symptom onset.

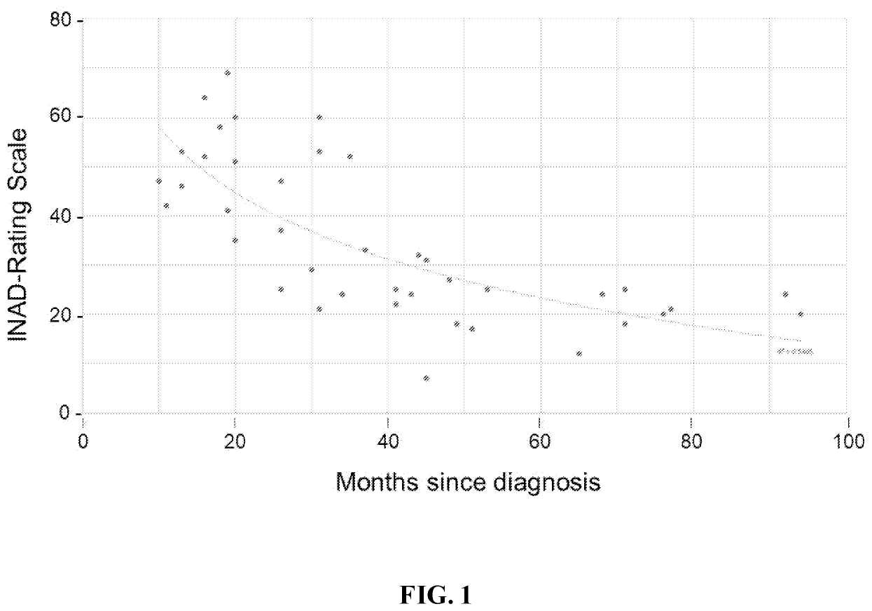

FIG. 1 is a graph of the general age-dependent decrease in the INAD rating scale score as a function of time since diagnosis. The expected progression of a given patient is not yet available from serial observations. However, the general age-dependent decrease in the INAD rating scale score is known (Atwal, Midei et al. 2020). While this is typically not the exact format needed to assess expected progression for clinical trials, it provides a general sense that in young patients, there is a rapid progression of about 0.47 units per month but with slower progression with increasing age.

Based on 28 subjects of European and Middle Eastern origin, most patients had delays in developmental milestones such as walking or speech. (Altuame, Foskett et al. 2020). For example, delineated age dependence of subjects losing specific skills such as sitting unsupported (median=33.5 months; range 24-57 months) and requiring placement of a nasogastric tube (median=6.5 years; range 2.6-12 years).

Deuterated linoleic acid (RT001; Retrotope) has been used in subjects with INAD (Adams, Midei et al. 2020). The compound is resistant to fatty acid oxidation and was shown to preserve mitochondrial function in cellular and Drosophila models of INAD.

### PLA2G6 Gene

The phospholipase 2 type VI protein gene (PLA2G6) resides on chromosome 22.

As detailed in Table 1 below, alternate splicing results in distinct mRNAs that encode either membrane-bound or cytoplasmic isoforms of the protein. RNA-Seq shows the gene is expressed in all tissues but at various levels from 2 to 20 adjusted reads per million.

TABLE 1
PLA2G6 gene, mRNA and protein isoforms.

| | Isoform | GenBank | Size | | Comment |
|---|---|---|---|---|---|
| Gene | | NG_007094 | 70,336 | bp | Chromosome |
| | | | | | 22q13.1 |
| mRNA | Isoform 1 | NM_001349864 | 3,295 | nt | N/A |
| | Isoform 2 | NM_001004426 | 3,137 | nt | N/A |
| Proteins | Isoform 1 | NP_001336793 | 806 | aa | Membrane bound |
| | Isoform 2 | NP_001004426 | 752 | aa | Cytoplasmic |

Phospholipase A2 type VI catalyzes the release of fatty acids from phospholipids. Phospholipases A2 catalyze hydrolysis of the sn-2 acyl-ester bonds in phospholipids, leading to the release of arachidonic acid and other fatty acids (Forsell, Olsson et al. 2005). PLA2G6 is a calcium-independent PLA2. Without being bound to a mechanism of action, there is no definitive biochemical mechanism linking low PLA2G6 enzyme activity and pathological findings in INAD. One prominent hypothesis posits that PLA2G6 deficiency leads to mitochondrial dysfunction with downstream cytotoxic effect on cells with high energy needs, such as neurons, (Kinghorn, Castillo-Quan et al. 2015). In addition, other critical functions of PLA2G6 have been demonstrated, such as a role in calcium signaling in astrocytes (Strokin, Seburn et al. 2012), retromer function (Lin, Lee et al. 2018) and ferroptosis (Beharier, Tyurin et al. 2020). Lin et al. (2018) have shown that PLA2G6 stabilizes key retromer proteins (Vps35 and Vps26). This affects endolysosomal trafficking, leading to a lysosomal expansion and severe elevation of ceramides. Treatment with a drug that lowers ceramide levels, myriocin, significantly improves the neurodegenerative phenotype in the Drosophila model. However, the potential utility of AAV9 mediated gene therapy as described here is independent of the exact molecular basis of disease pathogenesis as it may reverse the underlying cause for any of these proposed disease mechanisms.

Homozygous mutations in the PLA2G6 cause a range of genetic disorders collectively called PLAN (PLA2G6-associated neurodegeneration). The most severe is INAD (Infantile neuroaxonal dystrophy) which is an early onset neurodevelopmental disease affecting cognition, development, cognition and motor function.

There are multiple forms of the PLA2G6 cDNA encoding subtly different protein, with are at least 7 different versions of the mRNA. The major isoform, variant 1 (GenBank #NM_003560.4) encodes a 806 amino acid long PLA2G6 protein called isoform 1 which is membrane bound (SEQ ID NO: 1). But ˜20% of the mRNA is spliced in different way leading to the omission of exon 9. This results in a shorter protein called isoform 2 which is 752 amino acids long and is cytoplasmic. The existence of two isoforms of PLA2G6 is well conserved across many species from humans to mice to zebra fish.

### Exemplary Expression Cassette

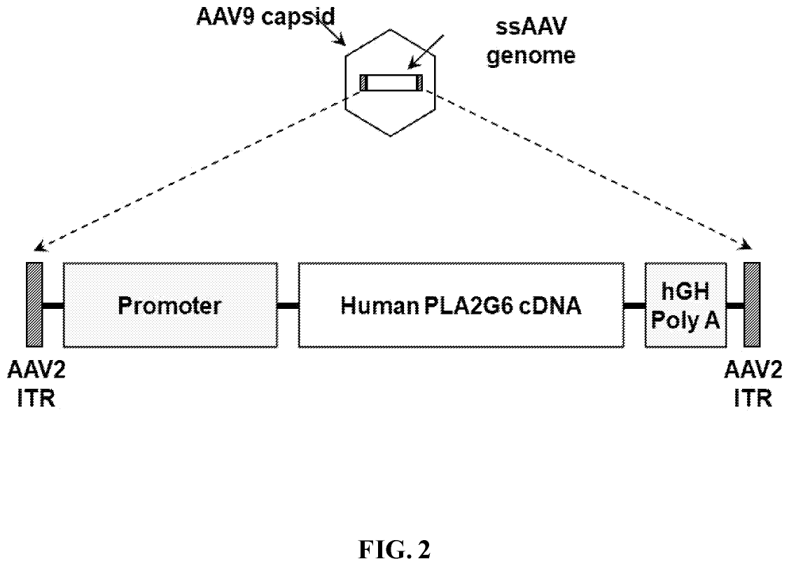

FIG. 2 is a schematic of the therapeutic entity of the invention, referred to AAV9-hPLA2G6. The promoter may be an EF1a promoter or SYN1 promoter depending of the result of in vivo efficacy studies in mouse model. The nucleic acid encoding the PLA2G6 gene may encode any copy of the PLA2G6 that restores a mutation in the PLA2G6 gene to wild-type functionality of the gene. The nucleic acid encoding the PLA2G6 gene may encode less than the entire gene, for example a portion of the PLA2G6 gene. In aspects of the invention, the nucleic acid sequence encoding the PLA2G6 gene may be SEQ ID NO. 1. Advantageously, the expression cassette does not comprise a woodchuck hepatitis virus post-transcriptional regulatory element (WPRE) between the nucleic acid sequence encoding a PLA2G6 gene and the poly(A) signal. For example, the entirety of the expression cassette may not comprise a woodchuck hepatitis virus post-transcriptional regulatory element.

### Adeno Associated Virus Vector

AAVs are particularly appropriate viral vectors for delivery of genetic material into mammalian cells. AAVs are not known to cause disease in mammals and cause a very mild immune response. Additionally, AAVs are able to infect cells in multiple stages whether at rest or in a phase of the cell replication cycle. Advantageously, AAV DNA is not regularly inserted into the host's genome at random sites, reducing the oncogenic properties of this vector.

AAVs have been engineered to deliver a variety of treatments, especially for genetic disorders caused by single nucleotide polymorphisms (“SNP”). Genetic diseases that have been studied in conjunction with AAV vectors include Cystic fibrosis, hemophilia, arthritis, macular degeneration, muscular dystrophy, Parkinson's disease, congestive heart failure, and Alzheimer's disease. The AAV can be used as a vector to deliver engineered nucleic acid to a host and utilize the host's own ribosomes to transcribe that nucleic acid into the desired proteins. See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). AAVs have some deficiency in their replication and/or pathogenicity and thus can be safer that adenoviral vectors. In some embodiments, the AAV can integrate into a specific site on chromosome 19 of a human cell with no observable side effects. In some embodiments, the capacity of the AAV vector, system thereof, and/or AAV particles can be up to about 4.7 kb. The AAV vector or system thereof can include one or more engineered capsid polynucleotides described herein.

AAVs are small, replication-defective, nonenveloped viruses that infect humans and other primate species and have a linear single-stranded DNA genome. Naturally occurring AAV serotypes exhibit liver tropism. As a result, transfection of non-liver tissue with traditional AAV vectors is impeded by the virus's natural liver tropism. Moreover, because the liver acts to break down substances delivered to a subject, transfection of non-liver tissue with unmodified AAV vectors requires higher dosing to provide sufficient viral load to overcome the liver and reach non-liver tissue. More than 30 naturally occurring serotypes of AAV are available. Many natural variants in the AAV capsid exist. AAV serotypes include, but are not limited to, AAV serotypes AAV1, AAV2, AAV3, AAV3B, AAV4, AAV5, AAV6, AAV7, AAV8, AAV9, AAV10, AAV11, AAV 12, AAV13. AAVs may be engineered using conventional molecular biology techniques, making it possible to optimize these particles, for example, for cell specific delivery, for minimizing immunogenicity, for tuning stability and particle lifetime, for efficient degradation, for accurate delivery to the nucleus. AAV vectors can be specifically targeted to one or more types of cells by choosing the appropriate combination of AAV serotype, promoter, and delivery method.

The AAV vector or system thereof may include one or more regulatory molecules, such as promoters, enhancers, repressors and the like. In some embodiments, the AAV vector or system thereof can include one or more polynucleotides that can encode one or more regulatory proteins. In some embodiments, the one or more regulatory proteins can be selected from Rep78, Rep68, Rep52, Rep40, variants thereof, and combinations thereof. In some embodiments, the muscle specific promoter can drive expression of an engineered AAV capsid polynucleotide.

The AAV vector or system thereof can include one or more polynucleotides that can encode one or more capsid proteins, such as the engineered AAV capsid proteins described elsewhere herein. The engineered capsid proteins can be capable of assembling into a protein shell (an engineered capsid) of the AAV virus particle. The engineered capsid can have a cell-, tissue-, and/or organ-specific tropism.

The AAV vector or system thereof can be configured to produce AAV particles having a specific serotype. In some embodiments, the serotype can be AAV-1, AAV-2, AAV-3, AAV-4, AAV-5, AAV-6, AAV-8, AAV-9 or any combinations thereof. In some embodiments, the AAV can be AAV1, AAV-2, AAV-5, AAV-9 or any combination thereof. One can select the AAV of the AAV with regard to the cells to be targeted; e.g., one can select AAV serotypes 1, 2, 5, 9 or a hybrid capsid AAV-1, AAV-2, AAV-5, AAV-9 or any combination thereof for targeting brain and/or neuronal cells; and one can select AAV-4 for targeting cardiac tissue; and one can select AAV-8 for delivery to the liver. Thus, in some embodiments, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In some embodiments, an AAV vector or system thereof capable of producing AAV particles capable of targeting cardiac tissue can be configured to generate an AAV particle having an AAV-4 serotype. In some embodiments, an AAV vector or system thereof capable of producing AAV particles capable of targeting the liver can be configured to generate an AAV having an AAV-8 serotype. See also Srivastava. 2017. Curr. Opin. Virol. 21:75-80.

In some embodiments, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the 2nd plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5. It will be appreciated that wild-type hybrid AAV particles suffer the same specificity issues as with the non-hybrid wild-type serotypes previously discussed.

Advantages achieved by the wild-type based hybrid AAV systems can be combined with the increased and customizable cell-specificity that can be achieved with the engineered AAV capsids can be combined by generating a hybrid AAV that can include an engineered AAV capsid described elsewhere herein. It will be appreciated that hybrid AAVs can contain an engineered AAV capsid containing a genome with elements from a different serotype than the reference wild-type serotype that the engineered AAV capsid is a variant of. For example, a hybrid AAV can be produced that includes an engineered AAV capsid that is a variant of an AAV-9 serotype that is used to package a genome that contains components (e.g., rep elements) from an AAV-2 serotype. As with wild-type based hybrid AAVs previously discussed, the tropism of the resulting AAV particle will be that of the engineered AAV capsid.

In some embodiments, the AAV vector or system thereof is configured as a “gutless” vector, similar to that described in connection with a retroviral vector. In some embodiments, the “gutless” AAV vector or system thereof can have the cis-acting viral DNA elements involved in genome amplification and packaging in linkage with the heterologous sequences of interest (e.g., the engineered AAV capsid polynucleotide(s)).

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Application publication No. US 2004-0171156 A1. Other suitable methods and techniques are described elsewhere herein.

Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vector described herein. AAV vectors are discussed elsewhere herein.

In some embodiments, the vector can have one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

### Promoter

Viral vectors may be limited in their ability to target transgene expression to specific cell populations, for example neuronal populations. This generally cannot be overcome by the use of tissue-specific promoters, as most are too large to be used with current viral vectors and expression from these promoters is often relatively weak.

Without being bound to a mechanism of action, expression of the cassette in all tissue types may be facilitated by the promoter in the cassette of the invention. For example, the vector may be expressed in all tissue types, including neuronal and muscular tissue, through the use of a SYN1 promoter or an EF1a promoter. Notably, the expression cassettes of the invention generally do not comprise a woodchuck hepatitis virus posttranscriptional regulatory element (WPRE), frequently paired with SYN1 and EF1a promoters.

### SYN1 Promoter

The small, 495-bp human synapsin 1 (SYN1) promoter has been shown to drive neuron-specific expression. Characterization of the 5′ flanking region of SYN1 has revealed a binding motif similar to neuron-restrictive silencer elements (NRSE), originally identified within type II sodium channels. Without being bound to a mechanism of action, the NRSE motif has been defined as a negative-acting regulatory element that prevents the expression of genes in non-neuronal cell types. In non-neuronal cells the NRSE is bound by the neuron-restrictive silencer factor (NRSF), a novel zinc-finger transcription factor that is only expressed at high levels in non-neuronal cells. Thus, the SYN1 promoter mediates expression to neuronal cells because the NRSF is absent.

### EF1a Promoter

Human elongation factor-1 alpha (EF1a) is a constitutive promoter of human origin that can be used to drive ectopic gene expression in various in vitro and in vivo contexts. EF1a is often useful in conditions where other promoters (such as CMV) have diminished activity or have been silenced (as in embryonic stem cells). EF1A promoters are consistent strong in all the cell types.

### Pharmaceutical Composition

Some embodiments of the invention may include any acceptable form of providing the AAV vector to a subject. For example, the AAV vector may be provided to the subject in the form of a composition or formulation comprising the AAV vector. The expression vector of this invention can be formulated and administered to treat a variety of disease states by any means that produces contact of the active ingredient with the agent's site of action in the body of the subject. The compositions, polynucleotides, polypeptides, particles, cells, vector systems and combinations thereof described herein can be contained in a formulation, such as a pharmaceutical formulation. In some embodiments, the formulations can be used to generate polypeptides and other particles that include one or more muscle-specific targeting moieties described herein. In some embodiments, the formulations can be delivered to a subject in need thereof. In some embodiments, component(s) of the engineered AAV capsid system, engineered cells, engineered AAV capsid particles, and/or combinations thereof described herein can be included in a formulation that can be delivered to a subject or a cell. In some embodiments, the formulation is a pharmaceutical formulation. One or more of the polypeptides, polynucleotides, vectors, cells, and combinations thereof described herein can be provided to a subject in need thereof or a cell alone or as an active ingredient, such as in a pharmaceutical formulation. As such, also described herein are pharmaceutical formulations containing an amount of one or more of the polypeptides, polynucleotides, vectors, cells, or combinations thereof described herein. In some embodiments, the pharmaceutical formulation can contain an effective amount of the one or more of the polypeptides, polynucleotides, vectors, cells, and combinations thereof described herein. The pharmaceutical formulations described herein can be administered to a subject in need thereof or a cell.

In some embodiments, the amount of the one or more of the polypeptides, polynucleotides, vectors, cells, virus particles, nanoparticles, other delivery particles, and combinations thereof described herein contained in the pharmaceutical formulation can range from about 1 μg/kg to about 10 mg/kg based upon the bodyweight of the subject in need thereof or average bodyweight of the specific patient population to which the pharmaceutical formulation can be administered. The amount of the one or more of the polypeptides, polynucleotides, vectors, cells, and combinations thereof described herein in the pharmaceutical formulation can range from about 1 μg to about 10 g, from about 10 nL to about 10 ml. In embodiments where the pharmaceutical formulation contains one or more cells, the amount can range from about 1 cell to 1×10², 1×10³, 1×10⁴, 1×10⁵, 1×10⁶, 1×10⁷, 1×10⁸, 1×10⁹, 1×10¹⁰ or more cells. In embodiments where the pharmaceutical formulation contains one or more cells, the amount can range from about 1 cell to 1×10², 1×10³, 1×10⁴, 1×10⁵, 1×10⁶, 1×10⁷, 1×10⁸, 1×10⁹, 1×10¹⁰ or more cells per nL, μL, mL, or L.

In embodiments, were engineered AAV capsid particles are included in the formulation, the formulation can contain 1 to 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, or 1×10²⁰ transducing units (TU)/mL of the engineered AAV capsid particles. In some embodiments, the formulation can be 0.1 to 100 mL in volume and can contain 1 to 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, or 1×10²⁰ transducing units (TU)/mL of the engineered AAV capsid particles.

### Pharmaceutically Acceptable Carriers and Auxiliary Ingredients and Agents

In embodiments, the pharmaceutical formulation containing an amount of one or more of the polypeptides, polynucleotides, vectors, cells, virus particles, nanoparticles, other delivery particles, and combinations thereof described herein can further include a pharmaceutically acceptable carrier. Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active composition.

In some embodiments, the pharmaceutical formulations described herein may be in a dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavernous, intrathecal, intravitreal, intracerebral, gingival, subgingival, intracerebroventricular, and intradermal. Such formulations may be prepared by any method known in the art.

Dosage forms adapted for oral administration can be discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as foam, spray, or liquid solution. In some embodiments, the oral dosage form can contain about 1 ng to 1000 g of a pharmaceutical formulation containing a therapeutically effective amount or an appropriate fraction thereof of the targeted effector fusion protein and/or complex thereof or composition containing the one or more of the polypeptides, polynucleotides, vectors, cells, and combinations thereof described herein. The oral dosage form can be administered to a subject in need thereof.

Where appropriate, the dosage forms described herein can be microencapsulated.

The dosage form can also be prepared to prolong or sustain the release of any ingredient. In some embodiments, the one or more of the polypeptides, polynucleotides, vectors, cells, and combinations thereof described herein can be the ingredient whose release is delayed. In other embodiments, the release of an optionally included auxiliary ingredient is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT (as sold by Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, the one or more of the polypeptides, polynucleotides, vectors, cells, and combinations thereof described herein can be formulated with a paraffinic or water-miscible ointment base. In some embodiments, the active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, the one or more of the polypeptides, polynucleotides, vectors, cells, and combinations thereof described herein is contained in a dosage form adapted for inhalation is in a particle-size-reduced form that is obtained or obtainable by micronization. In some embodiments, the particle size of the size reduced (e.g., micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active ingredient (e.g., the one or more of the polypeptides, polynucleotides, vectors, cells, and combinations thereof described herein and/or auxiliary active agent), which may be generated by various types of metered dose pressurized aerosols, nebulizers, or insufflators.

In some embodiments, the dosage forms can be aerosol formulations suitable for administration by inhalation. In some of these embodiments, the aerosol formulation can contain a solution or fine suspension of the one or more of the polypeptides, polynucleotides, vectors, cells, and combinations thereof described herein and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal, or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of one or more of the polypeptides, polynucleotides, vectors, cells, and combinations thereof described herein. In further embodiments, the aerosol formulation can also contain co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, or 3 doses are delivered each time.

For some dosage forms suitable and/or adapted for inhaled administration, the pharmaceutical formulation is a dry powder inhalable formulation. In addition to the one or more of the polypeptides, polynucleotides, vectors, cells, and combinations thereof described herein, an auxiliary active ingredient, and/or pharmaceutically acceptable salt thereof, such a dosage form can contain a powder base such as lactose, glucose, trehalose, mannitol, and/or starch. In some of these embodiments, the one or more of the polypeptides, polynucleotides, vectors, cells, and combinations thereof described herein is in a particle-size reduced form. In further embodiments, a performance modifier, such as L-leucine or another amino acid, cellobiose octaacetate, and/or metals salts of stearic acid, such as magnesium or calcium stearate.

In some embodiments, the aerosol dosage forms can be arranged so that each metered dose of aerosol contains a predetermined amount of an active ingredient, such as the one or more of the one or more of the polypeptides, polynucleotides, vectors, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas.

Dosage forms adapted for parenteral administration and/or adapted for any type of injection (e.g. intravenous, intraperitoneal, subcutaneous, intramuscular, intradermal, intraosseous, epidural, intracardiac, intraarticular, intracavernous, gingival, subgingival, intrathecal, intravitreal, intracerebral, and intracerebroventricular) can include aqueous and/or non-aqueous sterile injection solutions, which can contain anti-oxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and resuspended in a sterile carrier to reconstitute the dose prior to administration. Extemporaneous injection solutions and suspensions can be prepared in some embodiments, from sterile powders, granules, and tablets.

Dosage forms adapted for ocular administration can include aqueous and/or nonaqueous sterile solutions that can optionally be adapted for injection, and which can optionally contain anti-oxidants, buffers, bacteriostats, solutes that render the composition isotonic with the eye or fluid contained therein or around the eye of the subject, and aqueous and nonaqueous sterile suspensions, which can include suspending agents and thickening agents.

For some embodiments, the dosage form contains a predetermined amount of the one or more of the polypeptides, polynucleotides, vectors, cells, and combinations thereof described herein per unit dose. In some embodiments, the predetermined amount of the Such unit doses may therefore be administered once or more than once a day. Such pharmaceutical formulations may be prepared by any of the methods well known in the art.

### EXAMPLES

### Example 1: Non-Clinical Study for AAV Mediated Expression of Wildtype PLA2G6

Rational for Therapeutic Approach with AAV

In as much as INAD is a recessive genetic defect, gene replacement therapy may potentially provide a benefit. Mutations in PLA2G6 lead to reduced or absent phospholipase 2 type VI protein and resultant loss of the critical catalytic function. The challenge is to provide the missing gene to the correct cell types at the correct level. Sustained AAV-mediated expression of wildtype PLA2G6 is a rational approach to achieve this goal. Without being bound to a mechanism of action, the pathological hallmarks of INAD and electrodiagnostic testing clearly point to a neurological origin. In addition, data in mouse models clearly show that CSF delivery of a gene transfer vector was a highly effective route. Therefore, the plan is to deliver an AAV9 vector expressing wildtype PLA2G6 to the CSF of human subjects with INAD.

Intrathecal administration of either AAV9 or AAVrh. 10 vectors leads to successful infection of spinal neurons, dorsal root ganglia, and much of the brain, including the cerebellum and cortex. Not only does the intrathecal route provide direct access to the CNS, but it may be transduced in the presence of neutralizing anti-AAV humoral immunity (Gray, Nagabhushan Kalburgi et al. 2013). Accessing the brain through CSF rather than direct injection reduces procedure-related risk and inflammation (Rosenberg, Kaplitt et al. 2018). Intrathecal AAV administration through the lumbar spine or cisterna magna (ICM) has been used to treat multiple mouse models of disease, including Krabbe (Marshall, Issa et al. 2018), giant axonal neuropathy (Bailey, Armao et al. 2018), adrenomyeloneuropathy (Gong, Berenson et al. 2019), type II mucopolysaccharidosis, (Hinderer, Katz et al. 2016), Niemann Pick type C, (Kurokawa, Osaka et al. 2021), and familial amyotrophic lateral sclerosis (Li, Liu et al. 2017).

Scale-up of AAV delivery to CSF of non-human primates has also been investigated, providing important technical information. Injections of AAV in conjunction with radiological or MRI contrast agents allow the flow of vector following injection into the CSF to be assessed. Recent data in rhesus macaque show that the ICM route provides excellent access to both cerebrum and cerebellum with relatively poor access to the lower spinal cord (Piguet, de Saint Denis et al. 2021). Other groups have also assessed AAV distribution following intrathecal injection at the lumbar level. There is some suggestion that placing animals in the Trendelenburg position (supine with a 20° head-down tilt) may enhance vector distribution, but this is not a consensus ((Meyer, Ferraiuolo et al. 2015, Hinderer, Bell et al. 2018)).

Supporting the choice of ICM as route of delivery, a GLP compliant toxicology study of AAV9 gene transfer has been conducted in non-human primates (Hordeaux, Hinderer et al. 2018, Hordeaux, Hinderer et al. 2019). That study used 22 rhesus macaques with two doses of AAV9-IDUA expressing the human IDUA (alpha-L-iduronidase) gene. Some animals received concomitant immunosuppression. This study provides a rich resource of information relevant to treatment of INAD: 1) ICM injection with the methods and equipment applicable to humans can be conducted without serious procedure-related safety issues; 2) There were no hematological abnormalities observed related to ICM AAV9 administration; 3) There is a transient reaction to vector injection including transient pleocytosis in CSF consisting primarily of lymphocytes; 4) There was an anti-transgene immune response against the human IDUA gene despite 96.3% protein sequence identity with rhesus protein; 5) Anti-transgene and anti-vector immune responses were lessened by transient immunosuppression; 6) There were no histological changes in any organ except the dorsal root ganglia where a lymphocytic infiltrate was observed.

Additional confidence in the viability of the intrathecal approach to AAV9 gene therapy is provided by clinical data. There are multiple ongoing clinical trials with intrathecal AAV9 administration, including studies on spinal muscle atrophy (SMA) and giant axonal neuropathy (GAN). In the case of SMA, there was a proposed dose escalation from an initial dose of 6×10¹³ gc to 2.4×10¹⁴ gc (Clinical trials.gov registration NCT03381729). In the case of GAN, a maximum dose of 1.8×10¹⁴ was proposed (Clinicaltrials.gov registration NCT02362438). The proposed trial of AAV9 for Batten disease due to CLN7 mutations has a starting dose of 5×10¹⁴ vg (NCT04737460). Even when considering the differences in titration methods among the different programs, the human doses are very similar, indicating that dosing up to 5×10¹⁴ vg is deemed to be safe and well-tolerated by multiple groups using various methodologies.

INAD expression levels are high in corticospinal tracts, cerebellum, and hippocampus and that these areas are accessible by ICM injection of AAV9 vectors. The data suggests that ICM AAV9 injection is readily translatable to humans and that an acceptable safety profile may be anticipated. Therefore, the study will use the ICM route to assess efficacy in an INAD animal model, and proof-of-concept data is provided in the pre-IND background information package. That success, combined with the literature cited above, makes a convincing case for translation toward IND.

### Description of Nonclinical Studies

The therapeutic substance is an aqueous solution containing AAV9-hPLA2G6. This is a replication-defective recombinant adeno-associated virus serotype 9 vector expressing the human PLA2G6 cDNA for the membrane-bound protein isoform. The engineered genome includes the two inverted terminal repeats of AAV2 surrounding an expression cassette consisting of a promoter followed by the PLA2G6 cDNA (GenBank NM_001349864.2) and a polyadenylation signal from bovine growth hormone. The promoter may be the neuron-specific human SYN1 promoter or the ubiquitous EF1a promoter.

This design was informed by the studies carried out at University College London (Whaler 2018). The UCL vector is referred to herein as AAV9-PLA2G6 (UCL). Notably, the UCL vector comprised a woodchuck hepatitis virus post-transcriptional regulatory element (WPRE). The design was further informed by studies carried out at the laboratory of Hugo Bellen, Ph.D. at Baylor College of Medicine (BCM), using PLA2G6 gene driven by EF1a promoter.

Table 2, hereinbelow, provides a summary of these vectors (Table 2).

TABLE 1
Vectors used in pre-clinical studies

| | | | cDNA | |
|---|---|---|---|---|
| Study site | Vector | Promoter | (GenBank)⁴ | 3′ addition |
| UCL | AAV9- | Human SYN1 | | WPRE |
| | PLA2G6(UCL) | | | |
| BCM¹ | AAV9-EF1a- | Human EF1a | NM_001349864.2 | Human GH |
| | PLA2G6(BCM) | | | polyA, |

AAV9-PLA2G6 of the invention

| | | | | |
|---|---|---|---|---|
| Jackson - | AAV- | SYN1 and EF1a | NM_001199023.1 | Bovine GH |
| sponsored by | pSYN1/pEF1a- | | | polyA |
| INADcure² | mPLA2G6 | | | |
| Clinical study - | AAV9- | As determined | NM_001349864.2 | Human GH |
| Atlantic Health³ | hPLA2G6 | in mouse | | polyA |

¹Baylor College of Medicine (BCM) group also showed limited efficacy data with AAVPHP-eBb serotype. This vector was discontinued due to uncertainty of translating mouse data to non-human of primates (Mathiesen, Lock et al. 2020).
²For this study (the instant invention), mouse cDNA is used to provide maximum duration of transgene expression. Human and mouse proteins are 89.7% identical.
³This vector corresponds to that used in mouse efficacy studies but with the corresponding human cDNA. Comparability is established by 1) in vitro gene transfer with PLA2G6 activity assay. 2) in vivo assessment in mouse model.
⁴Based on rescue data in Drosophila and UCL gene therapy data, all cDNAs encode membrane bound isoform (isoform 1 in human).

### Mouse Models

Knockout mouse. The PLA2G6 knockout mice was generated by insertion of a neomycin cassette in place of exon 9. This is a null mutation based on both Northern (mRNA) and Western blots (protein) (Bao, Miller et al. 2004). Homozygous knockout mice have an average life span but reduced fertility. They exhibited rotarod and wire hanging defects at around 13 months of age.

Hypomorphic mouse. There is also a spontaneous mouse model with a defective endogenous retroviral insertion which occurred spontaneously in intron 1 upstream of the start codon (Strokin, Seburn et al. 2012). This model is a hypomorph with approximately 10% of normal PLA2G6 mRNA levels. Homozygous hypomorphs demonstrate a decrease in the grip strength test at 90 days of age, and their median survival is ˜120 days. This model is used in efficacy studies of the clinical entity AAV-hPLA2G6.

Missense mouse. Subsequently, another INAD mouse model, Pla2g6-INAD, has been established (Wada, Yasuda et al. 2009, Wada, Kojo et al. 2013). The Pla2g6-INAD mice bear a missense point mutation (G373R) in the ankyrin repeat domain of Pla2g6 generated by N-ethyl-N-nitrosourea mutagenesis. Homozygous G373R mice develop severe motor dysfunction and hematopoietic abnormality at a young age. The mice showed abnormal gait and poor motor performance as early as 7 to 8 weeks of age, detected by the hanging grip test. They survived a median of ˜100 days. Neuropathological examination revealed the widespread formation of spheroids containing tubulovesicular membranes similar to human INAD. Molecular and biochemical analysis revealed that the mutant mice expressed Pla2g6 mRNA and protein, but the mutated Pla2g6 protein had no glycerophospholipid-catalyzing enzyme activity.

The differences in phenotype between the three models described above are not fully accounted for. It seems counterintuitive that the null has more prolonged survival than the missense mutant. It is possible that a compensatory pathway is activated when there is no PLA2G6 in the early stage of development. Therefore, it may be important to not rely exclusively on survival and sensorimotor performance in one strain to assess therapies. Biochemical and morphological analyses may be added as proof-of-concept studies.

Compound Heterozygote. The studies conducted at BCM utilize a novel model based on the premise that residual PLA2G6 activity accelerates disease progression. This is a compound heterozygote (null/missense), and its use will provide additional confidence in the applicability of AAV gene transfer to treat the diverse genotypes of human PLA2G6 deficiency. In heterozygous mice bearing both the Pla2g6 knockout allele and Pla2g6-INAD (missense) allele, rotarod defects appeared slightly later than Pla2g6-INAD homozygotes but with much earlier onset (9 months earlier) than the Pla2g6 knockout homozygotes. The preliminary data show that AAV gene therapy is effective in this model. However, the BCM group also showed limited efficacy data with AAVPHP-eBb serotype. This vector was discontinued due to uncertainty of translating mouse data to non-human primates.

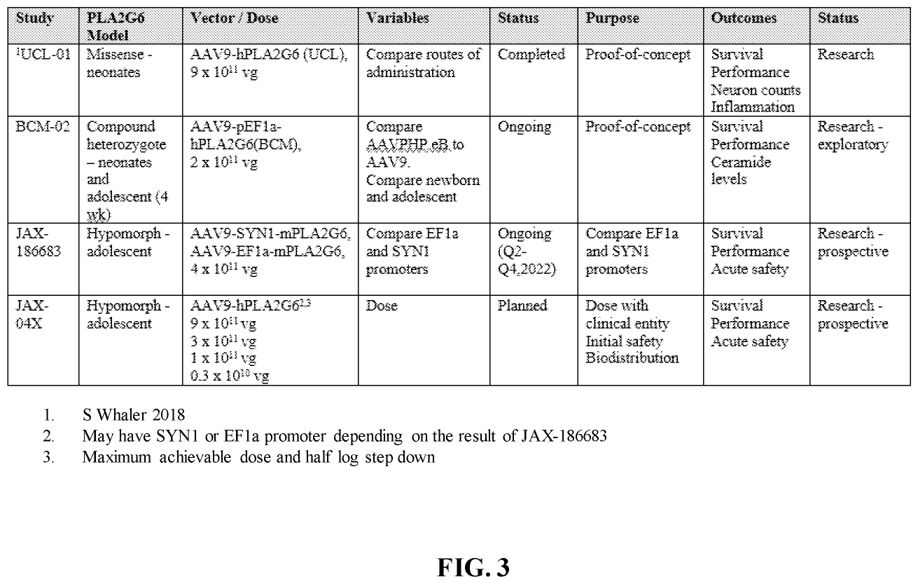

FIG. 3 provides Table 3, a summary of the detailed pre-clinical efficacy studies in mice. The diverse genotypes that give rise to PLAN and INAD in humans suggest that demonstrating efficacy in multiple mouse models will maximize credibility for a therapeutic entity.

### Treatment of Missense Mouse Model by ICV AAV9-hSYN1-hPLA2G6, UCL Study

Data published from Doctor of Philosophy thesis from the University College London (UCL) (Whaler 2018) provide an approximation of some of the pre-clinical data. The data establishes that CSF administration of an AAV9 vector expressing PLA2G6 cDNA using a SYN1 promoter may be a highly credible therapeutic candidate for treatment for INAD.

The UCL data consists of behavioral and immunohistochemical assays used to determine the therapeutic effect of AAV9-hPLA2G6 (UCL) when administrated by four different routes and compared to wild-type controls and untreated INAD mice. The routes of administration were: intracerebroventricular (ICV), a combined intracerebroventricular and intravenous delivery (ICV/IV), and a combined intracerebroventricular and intraperitoneal delivery (ICV/IP) and a single intravenous (IV) administration. The missense (G373R) mouse model was used for these studies, and homozygous knockouts were injected as neonates.

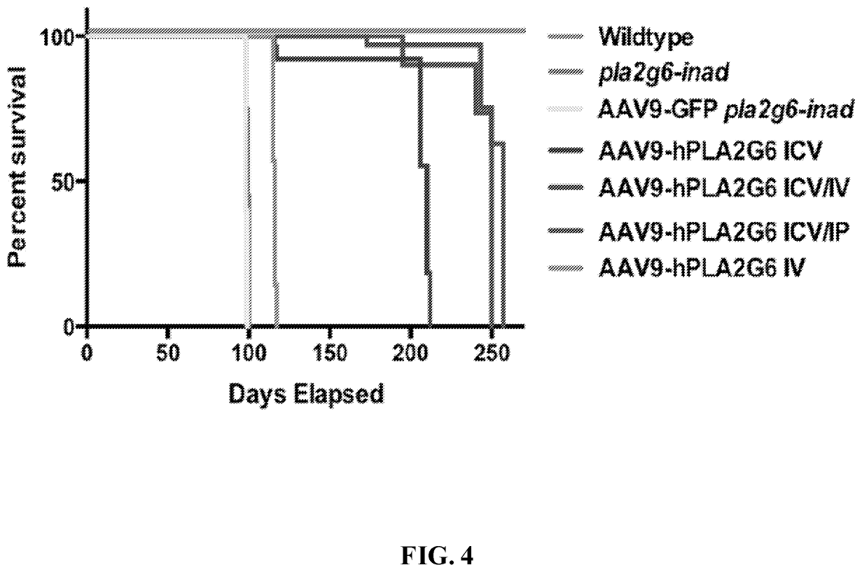

FIG. 4 is a graph of the impact of AAV9-hPLA2G6 (UCL) on survival in a missense mouse model of INAD. Cohorts of n=7 were injected by different routes as newborns and retained until humane sacrifice upon 10% loss of peak weight.

Untreated INAD mice had an average survival of 98 days, and there was no impact of AAV9-GFP injection on survival. However, neonatal AAV9-hPLA2G6 (UCL) by any route increased median survival. The intravenous route had the lowest impact, with average survival increased to 121 days. ICV injection had a greater impact, with an average survival of 215 days. An even greater impact was achieved by combining ICV with either intravenous or intraperitoneal injections for a survival of 255 days. The treated mice were sacrificed due to weight loss and did not show the clinical signs of INAD seen in untreated mice, such as posture and tremors.

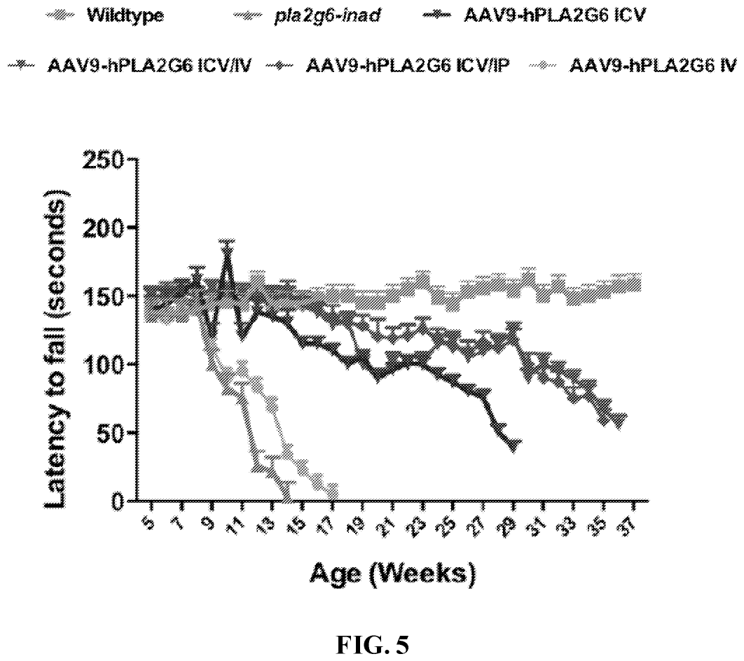

FIG. 5 is a graph of the impact of AAV9-hPLA2G6 (UCL) on rotarod latency in missense mouse model of INAD. Cohorts of n=7 were injected by different routes as newborns.

In all treated cohorts, behavioral analyses of pla2g6-inad mice revealed significant improvements in motor coordination in the AAV9-hPLA2G6 (UCL). Performance on the rotarod reflected the overall survival of mice treated by different routes of administration. The IV treated cohort had minimal improvement in performance over untreated mice, while ICV injection, with or without additional IP or IV injections, provided prolonged latency for the lifespan of the mice. Similar age-dependent improvements were observed in grip strength assay and open field test parameters where gene therapy treated pla2g6-inad mice were measured for distance, speed, and path efficiency.

PLA2G6 has essential roles in neuronal health, and region-specific progressive neuron loss is observed in the brains of untreated INAD mice.

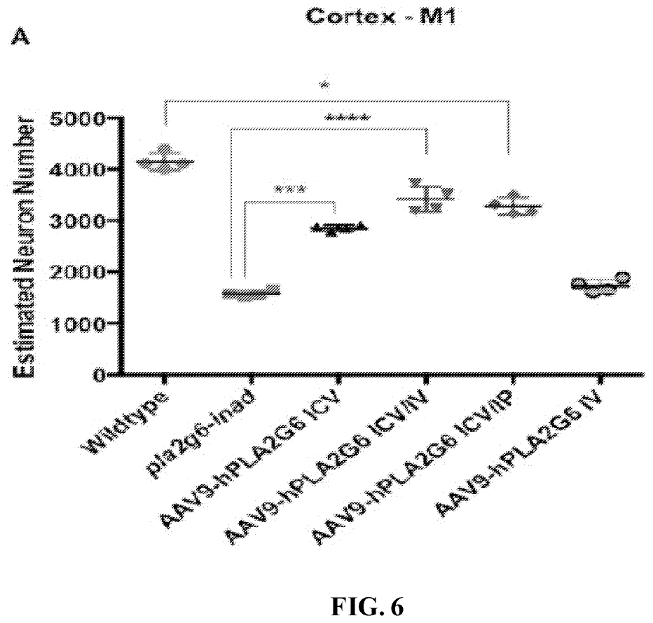

FIG. 6 is a graph of the impact of AAV9-hPLA2G6 (UCL) on neuron density. Unbiased stereological neuron counts were obtained from a series of NeuN stained sections of the M1 region of the cortex

The impact of neonatal AAV9-hPLA2G6 (UCL) administration was assessed by measuring neuron density in the motor cortex M1 region. The profound loss in end-stage INAD mice was alleviated by ICV injection of the AAV vector with further rescue by IVC+IV and ICV+IP injection. However, neuron counts were not fully normalized even after combined treatment in neonates. Similar rescue was detected in other regions of neuronal loss, including the ventral posteromedial nucleus and ventral posterolateral nucleus of the thalamus, the substantia nigra, and the brainstem.

AAV9 treated mice were also extensively assessed by morphological methods.

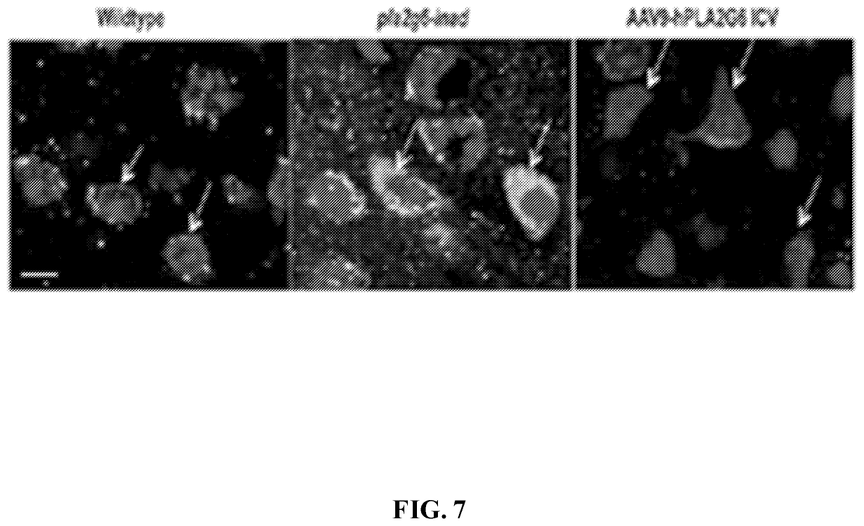

FIG. 7 is an image of the impact of AAV9-hPLA2G6 (UCL) on lysosomal function in neurons.

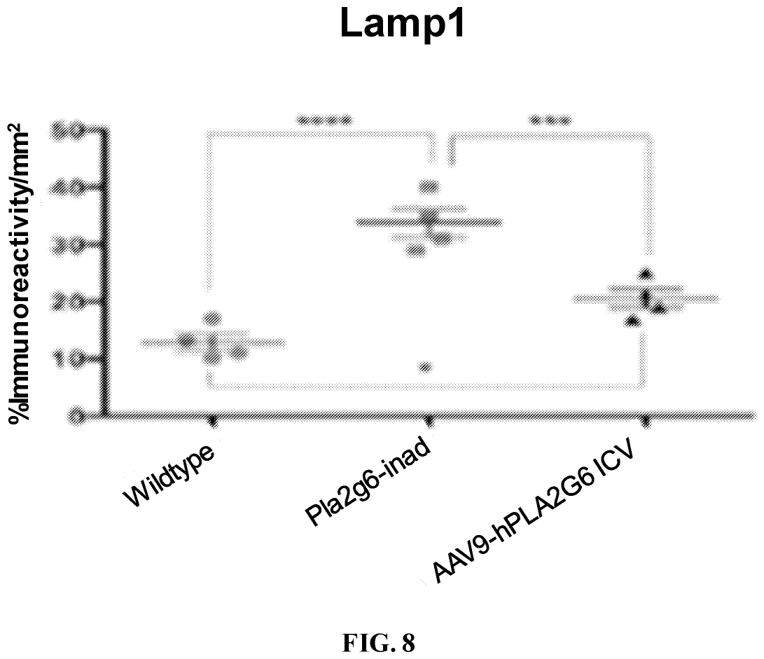

FIG. 8 is a graph of the impact of AAV9-hPLA2G6 (UCL) on lysosomal function in neurons.

As assessed by LAMP1 immunofluorescence, an increase in lysosomal immunoreactivity was observed in the brains of untreated pla2g6-inad mice. AAV9-hPLA2G6 (UCL) treated cohorts showed a significant reduction in lysosomal storage defect but did not reach wild-type levels.

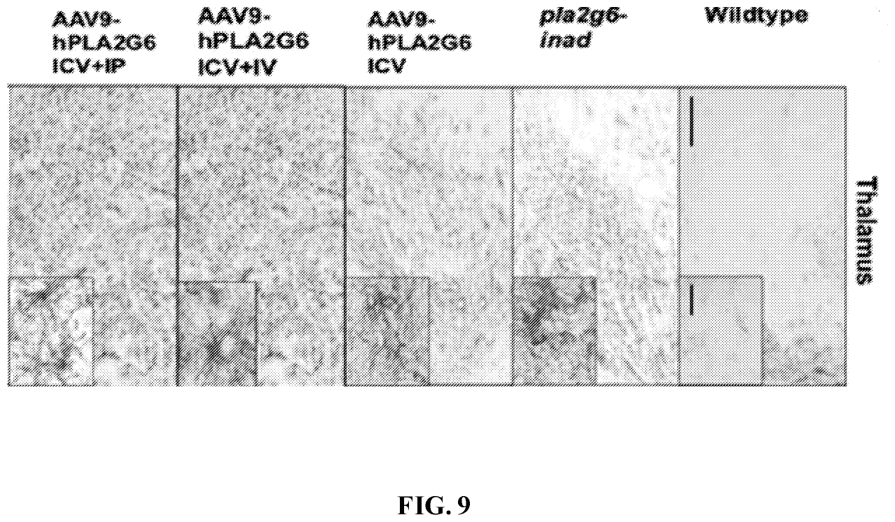

FIG. 9 is an image of the impact of AAV9-hPLA2G6 (UCL) on astrocyte activation. Astrocyte activation in thalamus of sections, stained for astroglial marker GFAP. Scale bar: 100 μm. Inserts show positively stained individual cells at higher magnification. Scale bar: 40 μm.

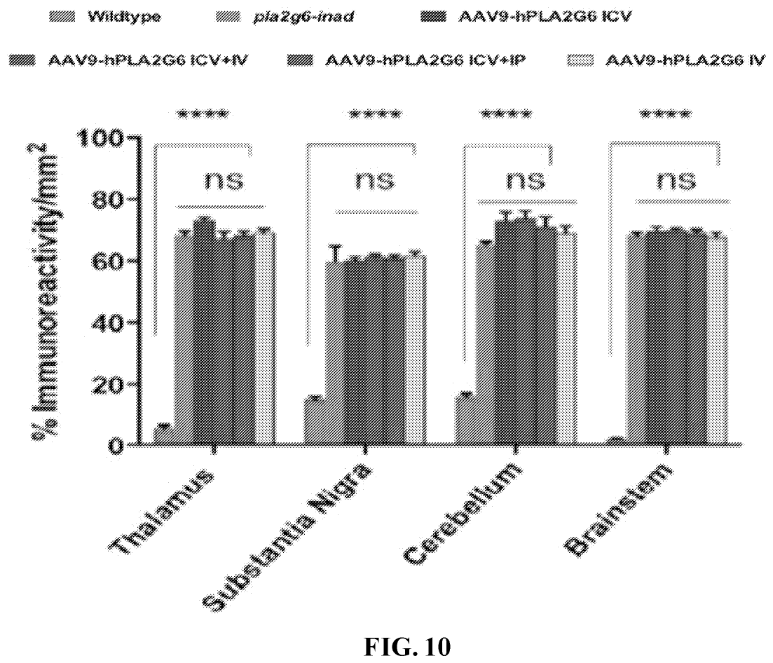

FIG. 10 is a graph of the impact of AAV9-hPLA2G6 (UCL) on astrocyte activation. Quantification of astrogliosis via immunoreactivity quantification of GFAP positive staining. Data represented as mean±S.D., compared by two-way ANOVA followed by Tukey's HSD test. ns—not significant, *p<0.05; **p<0.01; ***p<0.001; ****p<0.0001.

Assessment of glial fibrillary acidic protein (GFAP) as a marker of astrocyte activation in the missense mouse showed significant reactivity in multiple brain regions. Quantitative analysis by threshold analysis showed that AAV9-hPLA2G6 (UCL) gene therapy did not reduce immunoreactivity in astrocytes or microglia cells

While AAV9-hPLA2G6 (UCL) treatment provided improved survival and performance and resulted in neuronal rescue, the inflammatory responses observed in the brains of untreated pla2g6-inad mice remained present in the AAV9-hPLA2G6 (UCL) treated mice. It is possible that preferential neuronal expression of AAV9-hPLA2G6 (UCL) (for example, due to use of the SYN1 promoter in this study), means that cells such as astrocytes and microglia experienced continued neurodegeneration resulting in neuroinflammation. Additionally, the brain sections of the ICV gene therapy treated pla2g6-inad mice were processed and analyzed at their end-stage, which was at a significantly later time point compared to untreated pla2g6-inad mice.

### Treatment of Missense Mouse Model by AAV9-EF1a-hPLA2G6, BCM Study

The study being conducted at Baylor College of Medicine in the laboratory of Hugo Bellen, Ph.D. uses the compound heterozygous mouse model null/missense created by crossing heterozygous null mice to heterozygous missense mice. Thus, when vector is injected as newborns, 25% of injected mice have INAD and wildtype and heterozygous injected controls are also obtained.

This program is in its early stages, but limited data from pilot studies with AAV-PHP.eB-EF1a-hPLA2G6 suggests a positive impact of treatment.

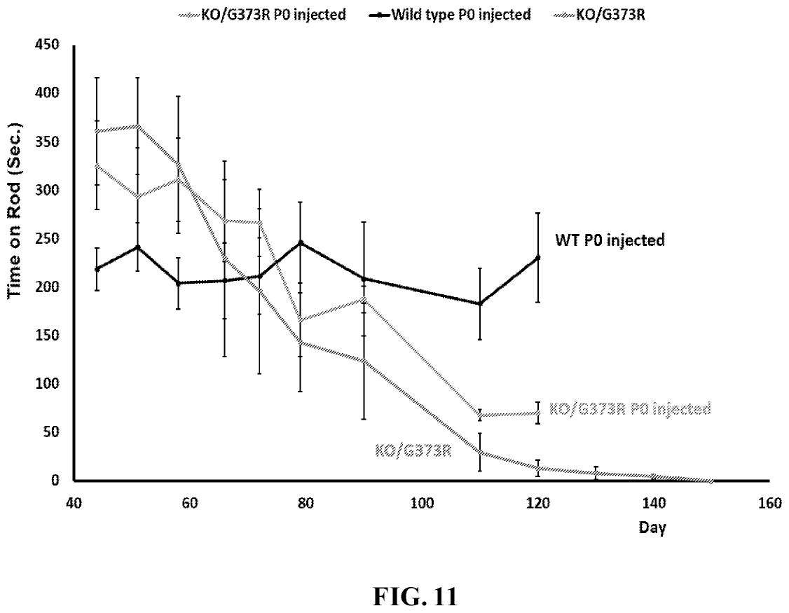

FIG. 11 is a graph of impact of AAV9-hPLA2G6 on rotarod latency in missense mouse model of INAD. Age-dependent rotarod latency for p0 ICV injection of AAV-PHP.eB-hPLA2G6 into null/missense compound heterozygous INAD mice. Means and standard error for: injected wildtype controls (n=4; black); injected INAD mice (n=5, blue) and untreated INAD (n=7 red).

Out of 26 pups 5 are PLA2G6 [KO/G373R]. These P0 injected animals are currently ˜120 days old. The P0 injected wild-type control animals do not display any rotarod defects, providing additional evidence that the injections are safe. The P0 injected mutant animals showed their first sign of decline in rotarod performance at 80-90 days, only ˜10 days later than the onset of the rotarod defects in the un-injected mutants. However, the performance of the injected mutants continued to deteriorate after ˜90 days with a minor but significant improvement when compared to un-injected mutants.

This data shows that the expression cassette with an EF1a promoter is functional in mice and reinforces that membrane-bound version of PLA2G6 is therapeutic. A similar study has been initiated with AAV9-EF1a-hPLA2G6 (BCM) vector which, depending on the result of study JAX-186683, may be the final clinical entity. The group of Dr. Bellen has shown that PLA2G6 functions to stabilize key retromer proteins so its absence results in endolysosomal trafficking which leads to a lysosomal expansion and severe elevation of ceramides. Ceramide levels are assessed in this experiment as a potential biomarker of disease progression.

Conclusions from UCL and BCM Studies

Publicly available data from UCL establishes that lifespan of the mouse model with INAD due to missense mutation in PLA2G6 can be successfully prolonged by ICV injection of AAV9 vector expressing PLA2G6 from a neuron specific promoter. Sensorimotor performance as assessed by multiple methods was enhanced in parallel with survival. There was protection against the regional loss of neurons seen in the untreated mutant mice and correction of lysosomal storage defect assessed by immunohistology. But there was no reduction of inflammatory response as assessed by GFAP staining.

This data from the UCL study establish that AAV9-PLA2G6 (UCL) is highly effective and results in doubling of lifespan following neonatal ICV administration in the INAD missense model. However, since the UCL vector as published has a woodchuck post-transcriptional element (WPRE) element, it is unsuitable for clinical translation based on prior published reports on possible oncogenic potential from this element (Kingsman, Mitrophanous et al. 2005).

Therefore, the present invention provides vectors lacking the WPRE, active in most cell types.

The mouse efficacy data in the previous section clearly establish that AAV9-hPLA2G6 (UCL) is a credible therapeutic. However, the following limitations are addressed by the present invention to provide a better basis for clinical translation:
1. Clinical entity. The entity used by UCL is AAV9-hPLA2G6 (UCL) which includes the WPRE element and the neuron-specific SYN1 promoter. In some contexts, WPRE increases mRNA stability and enhances transgene expression. However, the WPRE element after the PLA2G6 cDNA. is not advisable for clinical translation due to the possible oncogenic potential (Kingsman, Mitrophanous et al. 2005). The present invention shows that a vector, which lacks this element, will express PLA2G6 at a clinically sufficient level.
2. Route of administration. The best results came from combined ICV plus IV treatment. The incremental benefit on survival over ICV alone was 20% (215 days for ICV alone versus 259 days for combination). However, the combined route is not clinically feasible, and the maximum advantage was from ICV administration. Given the emerging clinical experience of ICM injection of AAV9 vectors, it is proposed that this will be the preferred route to access the CSF in human trials. (Human ICV injection would involve breaching the blood-brain barrier and is a complex and risky surgical procedure). Efficacy following ICM injection remains to be proven in mice; it is technically demanding procedure and ICV is a frequently used proxy.
3. Promoter. The definitive UCL data was derived from a vector with the SYN1 (neuron-specific) promoter. Notably, adding IV administration on top of ICV administration provided a 20% greater survival implying a possible benefit of PLA2G6 expression in non-CNS organs. CSF injection leads to leakage of vector in the circulation (Gray, Nagabhushan Kalburgi et al. 2013, Meyer, Ferraiuolo et al. 2015) and thereby non-CNS organs are also transduced. An even greater benefit may result from using EF1a promoter that expresses in all cell types.
4. Efficacy in adolescent mice. It is generally observed that maximum benefit following AAV gene transfer is achieved by neonatal injection, and efficacy declines with age of administration. The human study proposes administration to children, and so additional proof-of-concept studies will be conducted in adolescent mice (3 wk age) to match this population of patients.
5. Different mouse models. The UCL data is obtained in the missense model (G373R), which has a survival of ˜100 days when untreated. This is a convenient model for proof-of-concept, but there is uncertainty about whether it fully reflects pathogenesis in all human patients, at least some of which are nulls. Therefore, the studies use two different mouse models of INAD: the hypomorphic model and the compound heterozygous model (missense/null), to increase confidence that the therapy is universally applicable.
6. Biomarkers. INAD Natural history studies provide several clinically meaningful disease progression parameters, including a rating scale. While these may be good markers for the assessment of outcomes, it would also be desirable to have biomarkers of disease progression that apply to clinical trials.

### Study Design of JAX-18663. Treatment of Hypomorphic Model by ICV AAV9-mPLA2G6—Promoter Comparison

This study is conducted at a The Jackson Laboratory following a prospective study plan with full documentation of all mice which enter the study. The objectives are:
To show that the reengineered AAV vector lacking WPRE effectively prolongs life and improves performance in a mouse model of INAD.
To show impact in 3 wk old mice better reflecting the intended human population (versus neonatal mice)
To compare the impact of ICV injection of vectors with neuron-specific (SYN1) versus ubiquitous (EF1a) promoter. Without being bound to a mechanism of action, due to the expression of PLA2G6 in many tissues, there may be additional functions of the phospholipase protein beyond those in neurons that cannot be corrected by a vector with the neuron-specific SYN1 promoter. Leakage of the AAV9 vector with PLA2G1 expression driven by the ubiquitous EF1a promoter from the CSF following CSF administration may be sufficient to correct defects in non-CNS tissues.

TABLE 4
Design of study JAX-186683: Promoter comparison study.

| Cohort | Mice^{1, 2, 3} | Treatment | Dose | Route |
|---|---|---|---|---|
| 1 | N = 13 homozygous | None | N/A | N/A |
| 2 | N = 13 homozygous | ⁴AAV9-EF1a-mPLA2G6 | 1.8 × 10¹¹ vg | ICV |
| 3 | N = 13 homozygous | ⁴AAV9-SYN1-mPLA2G6 | 1.8 × 10¹¹ vg | ICV |
| 4 | N = 13 heterozygous⁵ | None | N/A | N/A |
| 5 | N = 13 heterozygous | AAV9-EF1a-mPLA2G6 | 1.8 × 10¹¹ vg | ICV |

¹B6.C3-Pla2g6m1J/CxRwb (Jackson labs Stock No: 019062; hypomorph). Randomized to study group and treated at 3 weeks of age.
²Interim sacrifice of n = 3 males at 28 days for serum chemistry, CBC, histopathology, vector biodistribution. Remaining n = 10 (5 each M and F) to be retained until humane sacrifice (20% weight loss).
³If possible, recover tissues for histopathology. Observation daily. General assessment weekly. Grip strength monthly, rotarod monthly.
⁴Both vectors at ~5 × 10¹³ vector genomes/ml to minimize aggregation. Sterile, low endotoxin vector. Dose comes from of maximum ICV injection volume (6 μl) and is lower than UCL data. Use of mouse gene is intended to decrease anti-transgene immunity and extend the duration of expression. Residual vector are retained for comparison to GLP and GMP material.
⁵Heterozygous littermates act as a control to assess long-term safety. Surviving heterozygous mice are sacrificed after 6 months.

Study Design JAX-04X. Final Dosing Study with the Therapeutic Entity

This study is conducted at a The Jackson Laboratory following a prospective study plan with full documentation of all mice which enter the study. The objectives are:
Dose dependence with final clinical vector-determine dose that achieves 90% maximum benefit to set target mid-dose for toxicology
By comparison with JAX-186683, determine if vector with human transgene is less effective in mice implying possible anti-transgene immune reaction.

TABLE 2
Design of study JAX-04X: Dose dependence with clinical entity

| Cohort | Mice | Treatment | Dose (Route) |
|---|---|---|---|
| 1 | N = 12 homozygous^{1, 2, 3} | None | N/A |
| 2 | N = 12 homozygous | ⁴AAV9-hPLA2G6 | 0.3 × 10¹¹ vg (ICV) |
| 3 | N = 12 homozygous | AAV9-hPLA2G6 | 1 × 10¹¹ vg (ICV) |
| 4 | N = 12 heterozygous | AAV9-hPLA2G6 | 3 × 10¹¹ vg (ICV) |
| 5 | N = 12 heterozygous | AAV9-hPLA2G6 | 9 × 10¹¹ vg (ICV) |

¹B6.C3-Pla2g6m1J/CxRwb (Jackson labs Stock No: 019062; hypomorph). Randomized to study group.
²n = 12 (6 each M and F) retained until humane sacrifice (20% weight loss).
³If possible, recover tissues for histopathology. Observation daily, General assessment weekly. Grip strength monthly, rotarod monthly.
⁴May have Syn1 or EF1a promoter depending in the result of JAX-186683. Maximum achievable dose and three dilutions of 3 fold each. Sterile, low endotoxin. Residual vector will be retained for comparison to GLP and GMP material.

### Pharmacology and Toxicology Data

A safety study is conducted by a GLP-compliant Contract Research Organization (CRO). In compliance with GLP expectations, animals will be randomized to the study group.

### Species and Strain

A toxicology study is performed in wild-type Sprague Dawley rats. Rats are chosen due to the ability to easily obtain sufficient blood for hematological analyses and the ease of ICM injection. The clinical study will involve pediatric subjects, to carry out our studies in young rats that will be 4 weeks old at the time of injection.

The inflammatory reaction in the dorsal root ganglia is a concern for the administration of AAV vectors by both intravenous and intrathecal routes. A retrospective review of this issue in 256 NHPs from 33 studies provides the overall context (Hordeaux, Buza et al. 2020). The pathology is minimal to moderate in most cases and characterized by mononuclear cell infiltrates, neuronal degeneration, and secondary axonopathy of central and peripheral axons on histopathological analysis. In a comprehensive study, the potential causes, including AAV serotype and purity, dose and route of administration, and recipient age, were investigated in primates. Older recipients with high doses of intrathecal AAV were most affected. There were no measurable clinical correlations, but nerve conduction velocity defects could be measured in severe cases. These inflammatory reactions are likely attenuated by the immunosuppression regimen now used in human studies to ensure long-term transgene expression following AAV gene transfer. These studies indicate caution in dosing and the need for monitoring spinal function in both toxicology and clinical studies related to INAD. This issue cannot be assessed in rats and has typically been studied in non-human primates (NHP).

The retrospective analysis (Hordeaux, Buza et al. 2020) established there is a dose-dependent DRG inflammation but that this is not transgene dependent except for foreign genes such as GFP. The clinical experience with ICM administration in multiple studies provides evidence of safety in humans and is the major consideration in the initial clinical dose. Also, the motor neuron function is impaired in INAD subjects, and the inflammatory response measured in wildtype NHP provide no indication of the possible impact in diseased INAD subjects.

### Test Article and Dose

The vector-delivered human PLA2G6 may be immunogenic in rats since rat and human proteins have 89.6% sequence identity. Toxicology using the human cDNA is assessed for the long-term expression of the human PLA2G6 transgene and the anti-vector and anti-transgene immune response.

Neonatal PLA2G6 missense mice in the BCM study were rescued with a 5 μl ICV injection of vector at 2×10¹³ vg/ml corresponding to a dose of 2×10¹³ vg/kg. Similarly study JAX-186683 uses a dose that corresponds to 1.5×10¹³ vg/kg. Pending a full dose-response in mice, we accept this target dose as the mid-dose for toxicology. To have a high dose cohort, we have calculated vector must be provided at ˜1×10¹⁴ gc/ml. This is achievable for AAV9 vectors in PBS with addition of 0.005% Pluronic 188 and 35 mM sodium chloride to inhibit aggregation. This will form the basis for our proposed vector formulation.

The test article is processed comparable to the proposed clinical vector and made in the same facility with the same reagents but with limited lot release testing.

### Cohort Design

Table 6 is a toxicology dose cohort for the study.

TABLE 3
Toxicology Dose Cohorts

| Cohort | Dose | Dose/kg |
|---|---|---|
| 1 | Vehicle control | N/A |
| 2 | AAV-hPLA2G6 | 2.5 × 10¹² gc/kg |
| 3 | AAV9-hPLA2G6 | 0.8 × 10¹³ gc/kg¹ |
| 4² | AAV9-hPLA2G6 | 2.5 × 10¹³ gc/kg |

¹Approximately the same as the proposed starting dose for the human phase 1 trial.
²Maximum available dose assuming vector concentration is 5 × 10¹³ gc/ml, 30 ul injection to 3 wk old rats (~60 g). Decrease doses by half log each for cohorts C and B.

Rats are injected by ICM route 30 ul of vector or vehicle. Since there is an approximately 90% success rate in ICM injection, additional rats built in over the n=5 per group are included in the final analysis. Each dose cohort will consist of n=24 rats of each sex, for a total of 192 rats for the entire study. Any rat that does not survive past the 4th day following vector administration is replaced. At 7, 30, and 120 days post-vector injection, a randomized subset of n=6 rats of each sex are sacrificed, leaving n=6 rats to replace any that die during the study.

Day 7 post-vector is chosen as the acute sacrifice time point when inflammatory changes mediated by the innate immune system may be evident. Acute reactions to high dose AAV injection have been observed at 6 to 8 days following injection (Meadows, Duncan et al. 2015).

Day 28 post-vector is chosen as an intermediate time point when inflammatory changes mediated by the acquired immune system may become evident. Studies on intravenous AAV in human adults suggest this is a critical time in terms of anti-vector and anti-transgene response (Nathwani, Tuddenham et al. 2011).

Long-term safety is established by sacrifice at 120 days post-vector. While there is some concern that neonatal rodents receiving intravenous AAV vectors may develop hepatocarcinoma up to 1 year of age, this is more common in newborn rodents or those undergoing extensive hepatocyte proliferation from liver regeneration (Dalwadi, Torrens et al. 2021).

### Assessments

In life assessments consist of
Cage-side observation twice daily: Animals are observed for morbidity, mortality, injury, and availability of food and water.
Detailed clinical observation weekly: Observations included evaluation of the skin, fur, eyes, ears, nose, oral cavity, thorax, abdomen, external genitalia, limbs and feet, respiratory and circulatory effects, autonomic effects such as salivation, nervous system effects including tremors, convulsions, reactivity to handling, and unusual behavior.
Weight: At least twice prior to randomization, daily post-viral vector delivery until Day 7, and once weekly thereafter.
Food Consumption: Weekly.

The safety measurements of this study are:
General observation including morbidity and mortality, weight gain, and food consumption.
Histopathological evaluation of H & E sections of the brain and other organs: For the brain, a total of 6 coronal sections will be assessed for each mouse as follows: 2 sections at −9.5 mm relative to Bregma providing an assessment of the cerebellum and underlying brainstem; 2 sections at −3.3 mm relative to the Bregma providing a view of the hippocampus, thalamus, and overlying cortex; 2 sections at +0.5 mm relative to Bregma providing a view of the striatum and overlying cortex. Other organ samples will be collected according to RITA guidelines (Ruehl-Fehlert, Kittel et al. 2003) with an examination of 2 paraffin sections per organ stained with hematoxylin and eosin for Colon, Diaphragm, Heart, Ileum, Kidney, Liver, Lungs, Lymph node (right brain-draining lymph node and any enlarged), Muscle (right quadriceps), Ovary, Prostate, Spinal cord (including DRGs and attached longitudinal nerve sections), Spleen, Testis, Uterus, and any gross lesions. A board-certified (ACVP) veterinary pathologist will evaluate all stained sections microscopically. All work, from tissue receipt through reporting, will be conducted in full accordance with Good Laboratory Practices (GLP) and a QA statement will accompany the draft(s) and final report. Histological processing, histopathological evaluation, and report will be contracted to a GLP compliant contractor with a board-certified veterinary histopathologist. Following the review of a draft histology report by the CRO conducting the toxicology study and IND holders, an audited report delineating all findings will be issued with additional studies if indicated.
Assessment of hematological and metabolic changes in blood/serum at the time of sacrifice.
Assessment of biodistribution of vector by qPCR under FDA sensitivity guidelines (<50 copies/μg in triplicate with and without spike). As mentioned above, some ICM injections may miss the target in CM. Such rats will have low vector in the spinal cord and therefore be distinguishable for correctly targeted rats.

Table 7 is a toxicology study schedule for the study.

TABLE 7
Toxicology Study Schedule

| | | Dose | | Study Groups³ | | | |
|---|---|---|---|---|---|---|---|
| Day¹ | Treatment | vg/kg² | Procedure | A⁴ | B⁴ | C⁴ | D⁴ |
| 0 | Vehicle (PBS) | 0 | ICM injection | 24M/24F | | | |
| 0 | AAV9 - | 5 × 10¹² | ICM injection | | 24M/24F | | |
| | CAG-hPLA2G6 | | | | | | |
| 0 | AAV9 - | 2 × 10¹³ | ICM injection | | | 24M/24F | |
| | CAG-hPLA2G6 | | | | | | |
| 0 | AAV9 - | Maximum | ICM injection | | | | 24M/24F |
| | CAG-hPLA2G6 | ~6 × 10¹³ | | | | | |
| Daily | | | Observation | All rats | All rats | All rats | All rats |
| Weekly | | | Weight, general exam | All rats | All rats | All rats | All rats |
| 7 | | | Blood/serum collection | 6M/6F | 6M/6F | 6M/6F | 6M/6F |
| | | | and Necropsy | | | | |
| 28 ± 2 | | | Blood/serum collection | 6M/6F | 6M/6F | 6M/6F | 6M/6F |
| | | | and Necropsy | | | | |
| 90 ± 2 | | | Blood/serum collection | 6M/6F | 6M/6F | 6M/6F | 6M/6F |
| | | | and Necropsy | | | | |

¹Day 0 represents rats of age 28 ± 2 days; and ~100 g of weight.
²The dose is delivered ICM in a volume of 20 ml.
³The study groups will be as follows: A. Vehicle control, PBS; B. Low dose; C. Target dose; D. Maximum feasible dose.
⁴24 rats of each sex are administered vector to allow for unexpected deaths. 6 randomly chosen rats of each sex will be analyzed at each time point.

### Example 2: Safety and Efficacy of Intrathecal Gene Transfer in Infantile Neuroaxonal Dystrophy (INAD) Using AAV9 Expressing Human PLA2G6 (Phase I/II Open-Label Trial)

### Objectives

To determine the safety of ICM injection of AAV9 vector expressing PLA2G6 in children with INAD.
To determine if an injection of AAV9-PLA2G6 at a safe dose can impact INAD disease progression as assessed by validated rating scales.

### Study Design

INAD is a rare disease with no current treatment options beyond attempting to alleviate symptoms. Since this is a pediatric disease with inevitable progression, the potential therapy is assessed in pediatric subjects. Therefore, a potential benefit is offered to study subjects, leading to an open-label phase 1 study with no simultaneous randomized control group. The ongoing natural history at the University of Oregon Health Sciences Center provides a large cohort of untreated subjects as a basis for comparison.

An open-label is conducted as a two-part study that establishes dose (Part A) and efficacy, safety, and tolerability (Part B) of AAV9-hPLA2G6. The objective is to demonstrate clinically meaningful changes in the INAD rating scale after a single, safe, and tolerated administration of AAV9-hPLA2G6.

The study has two parts:
Part A (dose escalation); age 1-12 years of age at Screening
Part B (dose expansion); age 1-12 years at Screening

Subjects are dosed with a single administration of AAV9-hPLA2G6 and followed for 5 years.

Guidance on dose selection (i.e., dose cohort expansion, dose-escalation) in Part A and dose selection for Part B (dose expansion) is based on Data Review evaluation of safety, tolerability, and preliminary data in treated subjects. A Data Safety Monitoring Board (DSMB) is constituted for this purpose. Three dose cohorts are formed during Part A with dosing of up to 5 subjects per dose cohort. 3 to 5 subjects are dosed in Cohort 1 of Part A and receive a single ICM administration of AAV9-hPLA2G6 at dose level 1 (tentatively 2×10¹⁴ vg). Based on interim review by DSMB after the first three subjects, an acceptable safety profile but absence of effect (stabilization of score on INAD rating scale) will trigger escalation to dose cohort 2 at a 2-fold higher dose level (4×10¹⁴ vg) for Cohort 2. By similar criteria, the dose may be escalated to dose level 3 (current target 6×10¹⁴ vg for Cohort 3). Data is reviewed for dose escalation after a minimum of 26 weeks of safety and efficacy data have been collected in all subjects in that cohort. There can be additional meetings to review data for dose expansion and dose selection for Part B as needed. Part B consists of expansion at the maximum tolerated dose to up to 15 subjects.

### Use of Pediatric Subjects

This previously untested therapeutic entity is ideally tested in subjects who fully understand and consent to the therapeutic limitations and risks (i.e., adults). Nevertheless, this study is only feasible in pediatric subjects for the following reasons:
The risk-benefit ratio is more favorable in pediatric subjects. The risks of ICM AAV9 usage are unknown, although there is clinical data known to the FDA on intrathecal AAV9 that is sufficient to justify the dose of AAV9 to treat SMA and GAN.
Anti-AAV9 immunity increases with age, and therefore immune-mediated blockade of transduction and adverse events are likely to be less in pediatric subjects.
The majority of individuals in our international registry are less than 18 years old, with most recently diagnosed cases being less than five years old.
Any benefit of gene therapy likely is to slow progression rather than reverse pathology. Therefore, the potential benefits to INAD patients, as with many neurodegenerative diseases, are greater in young subjects when the loss of neural structure and function has not progressed to an irreversible stage.
The ultimate target for treatment is pediatric subjects who can be enrolled immediately following diagnosis. Therefore, an informative study must establish safety and efficacy in this same population.
Adolescent mouse efficacy studies will establish feasibility in models that approximate pediatric patients.
Patients do not live to adult age.

### Vector Administration and Immunosuppression

Vector are administered to the ICM by radiologically guided injection. These methods are being extensively used for this purpose, and the details protocols, precautions, and materials are publicly available. Surgeons are trained by a group who have previously used this methodology. Compatibility with injection device will be established.

Immunosuppression will include 1) tacrolimus for 13 weeks at 0.1 mg/kg/day with target trough levels of 4-8 ng/ml and 2) prednisolone at 1 mg/kg/day for 13 weeks with a 3 week taper.

### Dose

The target human dose is established and dependent on data obtained from the instant study. The following is considered in selecting the target dose:
Dose-response in mice. Determine dose-response based on multiple outcome criteria and establish a target dose as the minimum dose that gives maximum achievable behavioral and histological impact.
It is possible that limitations on vector stability and aggregation at high concentrations combined with injection volume do not allow a plateau in therapeutic effect to be established in mice. In that case maximum, achievable dose based on injection volume and achievable vector concentration is the target.
The target dose will be scaled up to rats for toxicology and humans based on body weight. The scale-up calculation assumes a final human target dose of 6×10¹⁴ gc/kg for the sake of illustration. This is consistent with AAV9 doses used in other clinical trials, such as the target dose of 5×10¹⁴ vg for CLN7 gene therapy (clincaltrials.gov NCTC04737460).
A toxicology study will bracket the target dose and determine if there is an upper limit due to dose-limiting toxicity.
Human scale-up will also use any available human clinical data from intrathecal administrations of AAV9 vectors. Following dose escalation in clinical trials, a study of intrathecal administration of AAV9 to treat spinal muscular atrophy is now proposing 2.4×10¹⁴ gc total dose (Clinicaltrials.gov registration NCT03381729). Similarly, intrathecal administration of self-complementary AAV9 to treat giant axonal neuropathy proposes a dose escalation to 1.8×10¹⁴ gc (Clinicaltrials.gov registration NCT02362438).

Table 8 details the provisional scale up to Human Dose.

TABLE 8
Provisional Scale up to Human Dose

| | | | | | Injection |
|---|---|---|---|---|---|
| | Weight | Volume | Dose | | time |
| Species | (kg)³ | (ml) | (gc)¹ | Dose (gc/kg) | (minutes) |
| Mouse | 0.01 | 0.01 | 2 × 10¹¹ | 2 × 10¹³ | 0.1 |
| Rat² | 0.06 | 0.03 | Up to | Up to | 0.1 |
| | | | 1.5 × 10¹² | 2.5 × 10¹³ | |
| Human | 15 | 3 | 1-6 × 10¹⁴ | 7 × 10¹² to | 6 |
| INAD⁴ | | | | 4 × 10¹³ | |

¹Dose based on BCM study.
²For GLP toxicology - need vector at 5 × 10¹⁵ vg/ml.
³Neonatal mouse, 3 week old rat, 5 yr old human.
⁴Human starting dose is below dose/kg in rat safety study.

### Inclusion/Exclusion Criteria

### Inclusion Criteria

Subjects are eligible to be included in the study only if all of the following criteria apply:

### Age

Male and female patients aged 1-12 years at Screening are eligible to participate in this study.

### Type of Participant and Disease Characteristics

Proband must have a genetically confirmed diagnosis of INAD with bi-allelic pathogenic mutations in the PLA2G6 gene.
Willingness to abstain from hepatotoxic substances/medications from Screening through at least 52 weeks post-AAV9-hPLA2G6 administration.
Stable doses of medication for ≥4 weeks prior to Screening. Subjects must also be willing to maintain stable doses of these medications during Screening and throughout the study, unless the change is medically indicated.
Willingness to abstain from the donation of organs or tissue.
Proband meets age-appropriate institutional criteria for the use of sedation or anesthesia as needed.
The subject is up to date on childhood vaccines according to local guidelines.
Willingness and capability, per the investigator's opinion, to comply with study procedures and requirements.

### Informed Consent

Parent(s)/guardian(s) with the legal capacity to execute an IRB-approved consent for medical research must be able to participate in the consent process.
Parent(s) or guardian(s) must be willing and able to provide written, signed informed consent after the nature of the study has been explained and prior to the performance of any research-related procedure. Subjects under the age of majority who have appropriate comprehension must be willing and able to provide written assent (if required by local HA or the EC/IRB) after the nature of the study has been explained and prior to the performance of any research-related procedure.

### Exclusion Criteria

Subjects are excluded from the study if any of the following criteria apply:

### Medical Conditions at Screening or Enrollment

Any evidence of active infection (including SARS-COV-2) or immunosuppressive disorder, including HIV infection.
Prior liver biopsy or imaging showing significant fibrosis of 3 or 4 as rated on a scale of 0-4 on the Batts-Ludwig or METAVIR scoring systems, or an equivalent grade of fibrosis if an alternative scale is used.
History of malignancy (other than non-melanoma skin cancer) within 5 years of Screening, or any history of hepatic malignancy
Contraindication to use of corticosteroids (CS) or history of a condition that could worsen with CS therapy, as assessed and determined by the investigator
Any condition that, in the opinion of the investigator or Sponsor, would prevent the subject from fully complying with the requirements of the study (including possible corticosteroid treatment outlined in the protocol) and would impact or interfere with the subjects' safety evaluation and interpretation of subject safety or efficacy result.
Known allergy or hypersensitivity to AAV9-hPLA2G6 investigational product formulation

### Prior/Concomitant Therapies

Taking any medications chronic medications that are systemic immunosuppressive agents may be hepatotoxic and may reduce or increase the plasma concentration of corticosteroids. Exceptions must be approved by the medical monitor.
Prior treatment with gene therapy.
Treatment with any investigational product within 30 days prior to the Screening period.
Use of systemic immunosuppressive agents, including CS, within 30 days prior to AAV9-hPLA2G6 administration.
Immunization with live or live-attenuated vaccines within 30 days prior to Screening and/or immunization with other vaccines within 14 days prior to AAV9-hPLA2G6 administration.
Administration of certain vaccines is allowed throughout the study.

### Prior/Concurrent Clinical Study Experience

Concurrent enrollment in another clinical study, unless it is an observational (non-interventional) clinical study that does not interfere with the requirements of the current protocol or has the potential to impact the evaluation of efficacy and safety of AAV9-hPLA2G6 and with prior consultation with the Primary Investigator/Medical Monitor.

### Diagnostic Assessments

Detectable antibodies to the AAV9 capsid at Screening.
INAD-RS score of 5 (total on all parameters) or lower at Screening.
Significant liver dysfunction at Screening as defined by any of the following:
   ALT (alanine transaminase)>2 times the upper limit of normal
   AST (aspartate aminotransferase)>2 times the upper limit of normal
   GGT (gamma-glutamyltransferase)>2 times the upper limit of normal
   Bilirubin>2 times the upper limit of normal
   INR (international normalized ratio)≥1.5
   Subjects whose liver laboratory assessments fall outside these ranges may undergo repeat testing of the entire liver panel. If eligibility criteria are met on retest within the screening window, the subject may be enrolled after confirmation by the Medical Monitor.
Significant thrombocytopenia (platelet count<100×10⁹/L) at Screening.
Any prior infection with hepatitis B (HBV) as evidenced by positive serology testing (HBV surface antigen [HBsAg], HBV surface antibody [HBsAb], and/or HBV core antibody [HBcAb]) at Screening. Isolated HBsAb positivity for HBV vaccination, in conjunction with negative confirmatory HBV DNA testing at Screening is not exclusionary.
Any prior infection with hepatitis C (HCV) as evidenced by positive HCV antibody testing and confirmed by positive viral PCR RNA testing at Screening.
Active tuberculosis (TB) or untreated latent TB infection (LTBI), determined by a positive QuantiFERON test at Screening.
   Indeterminate QuantiFERON may be repeated once. It will be considered positive if retest results are positive or indeterminate.
QuantiFERON-positive subjects are excluded from the study unless they have documentation of completed chemoprophylaxis for LTBI prior to Screening.
Clinically significant liver disease as assessed by ultrasound at Screening.
Grade 3 or Grade 4 fibrosis as assessed by Fibroscan at Screening.
Serum creatinine≥1.5 mg/dL at Screening.
Hemoglobin A1C≥8.0% or glucose≥250 mg/dL at Screening.

### Other Exclusions

Major surgery planned during the Screening period through 52 weeks following AAV9-hPLA2G6 administration.
Subjects currently planning to receive an organ transplant.

### Disclosure Statement

This is an open-label treatment study with no blinding.

### Number of Subjects

Up to 25 subjects may be dosed in the study.

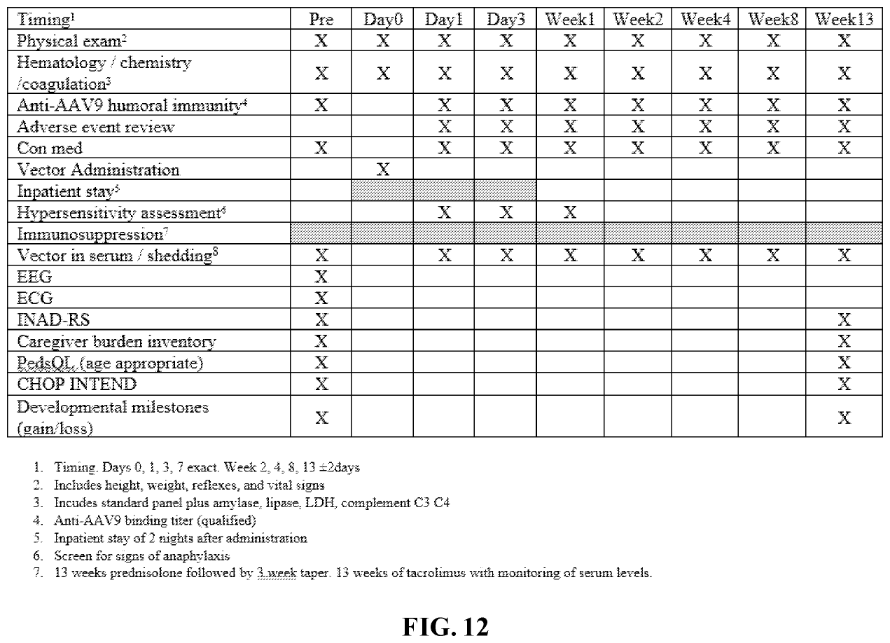

FIG. 12 is Table 9, a simplified schedule of short-term safety assessments.

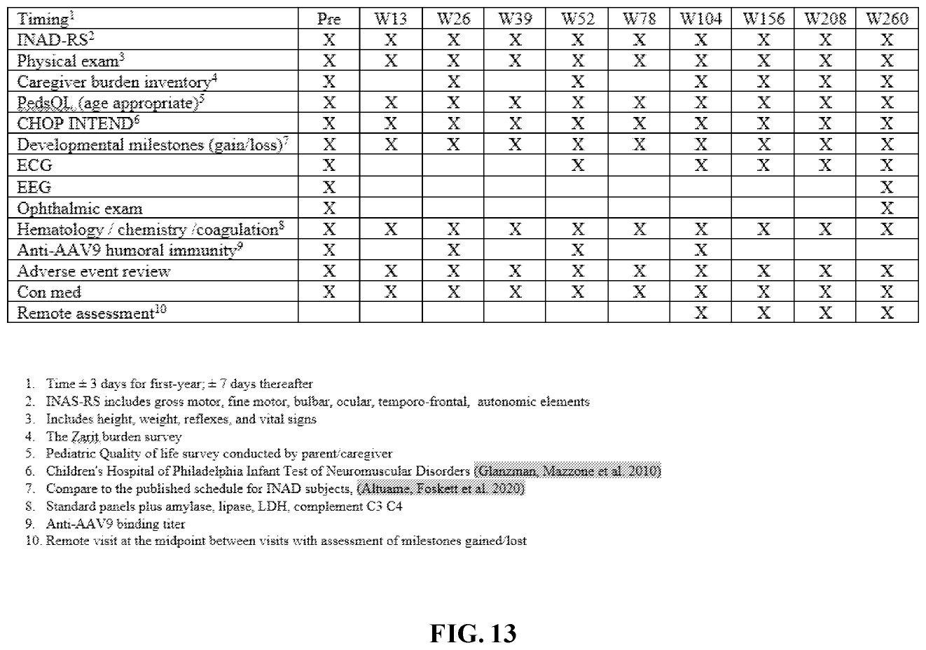

FIG. 13 is Table 10, a simplified schedule of long-term efficacy assessments.

### Outcome Parameters

### Efficacy

Proportion of subjects achieving stabilization or improvement of INAD rating scale by Week 26 post-infusion of AAV9-hPLA2G6 within each dose cohort.
Change in baseline of INAD rating scale over time
Change in baseline of CHOP Intend over time.
Change in baseline of the Hammersmith scale over time

### Safety

Treatment-emergent adverse events
Changes in standard clinical laboratory values
Changes in transaminase level
Immune response against the AAV capsid and PLAG26 transgene
Blood biodistribution and urine, stool, and saliva vector shedding assessment

### Secondary

Change in PedsQL Infant scale (ages 0-24 months) and PedsQ1 Neuromuscular module score (ages 2-12) from baseline at multiple points during the protocol per SoA
Change from baseline of Caregiver Burden scale over time as assessed by the Zarit Burden Interview or PedsQL Family Impact Module
Change developmental gains and developmental loss of skills over time

### Stopping Rules

If any of the following events during the study (Part A or Part B) who has received AAV9-hPLA2G6 administration, further enrollment and dosing is halted to complete an extensive safety analysis.
A subject with any SAE assessed as related to study drug (excluding infusion reactions CTCAE Grade 2 or lower).
A subject with any Grade 3 or higher adverse event (excluding ALT elevation) assessed as related to AAV9-hPLA2G6 administration
A subject with an AE of hepatic failure
Liver dysfunction (criteria do not apply to ALT elevations with an extra-hepatic etiology):
   ALT>5×ULN, for more than 2 weeks
   ALT>3×ULN and (total bilirubin>2×ULN or INR>1.5)
   ALT>3×ULN with signs and symptoms of liver dysfunction
A subject with an occurrence of any cancer (except non-melanoma skin cancer) after AAV9-hPLA2G6 administration and through the long-term follow-up period

### Discussion of Non-Clinical and Clinical Trials of AAV9-hPLA2G6

Results from Mouse Study

The results of administration of AAV9-hPLA2G6 in mice show that the reengineered AAV vector lacking WPRE effectively prolongs life and improves performance in a mouse model of INAD.

The results further show that impact in 3 wk old mice better reflecting the intended human population (versus neonatal mice).

The results further show that PLA2G1 expression driven by the ubiquitous EF1a promoter is sufficient to correct defects in non-CNS tissues.

Results from Human Clinical Study

The results of administration of AAV9-hPLA2G6 to humans show the safety and efficacy of administration of AAV9-hPLA2G6.

For example, the results show that a proportion of subjects achieved stabilization and improvement of INAD rating scale by Week 26 post-infusion of AAV9-hPLA2G6 within each dose cohort.

The results further show an improvement in baseline of INAD rating scale over time post-infusion of AAV9-hPLA2G6.

The results further show an improvement in CHOP Intend over time post-infusion of AAV9-hPLA2G6.

The results further show an improvement in in baseline of the Hammersmith scale over time post-infusion of AAV9-hPLA2G6.

The results further show an improvement in PedsQL Infant scale (ages 0-24 months) and PedsQ1 Neuromuscular module score (ages 2-12) from baseline at multiple points during the protocol per SoA post-infusion of AAV9-hPLA2G6.

The results further show an improvement from baseline of Caregiver Burden scale over time as assessed by the Zarit Burden Interview or PedsQL Family Impact Module post-infusion of AAV9-hPLA2G6.

The results further show an improvement in developmental gains and developmental loss of skills over time post-infusion of AAV9-hPLA2G6.

### Example 3: Hybrid cDNA/Genomic PLA2G6 Gene

Homozygous mutations in the PLA2G6 cause a range of genetic disorders collectively called PLAN (PLA2G6-associated neurodegeneration). The most severe is INAD (Infantile neuroaxonal dystrophy) which is an early onset neurodevelopmental disease affecting cognition, development, cognition and motor function.

With the intention of treating this disorder by gene therapy, AAV mediated gene delivery may be applicable providing an additional good copy of the gene to compensate for the two defective copies in a patient's cells. The whole PLA2G6 gene (intron plus exons) is over 70,000 nucleotides, much larger that the carrying capacity of AAV, which is typically ˜4,500 nucleotides. The use of cDNA in which introns are spliced out of the exon can reduce the size of the nucleic acid to be carried by the AAV vector.

There are multiple forms of the PLA2G6 cDNA encoding subtly different protein. There are at least 7 different versions of the mRNA. The major isoform, variant 1 encodes an 806 amino acid long PLA2G6 protein called isoform 1, which is membrane bound. However, ˜20% of the mRNA is spliced in different way leading to the omission of exon 9. This results in a shorter protein called isoform 2 which is 752 amino acids long and is cytoplasmic.

Conventional work on PLA2G6 gene studies have used AAV vectors with a cDNA encoding isoform 1, the most abundant isoform, in their studies. However, the existence of two isoforms of PLA2G6 is well conserved across many species from humans to mice to zebra fish. Without being bound to a mechanism of action, this strongly implies that the cytoplasmic isoform has some function independent of the membrane bound version isoform 1.

A gene therapy is developed by the present invention in which both isoforms are expressed from the AAV vector simultaneously. This is achieved by creating a cDNA/genomic DNA hybrid.

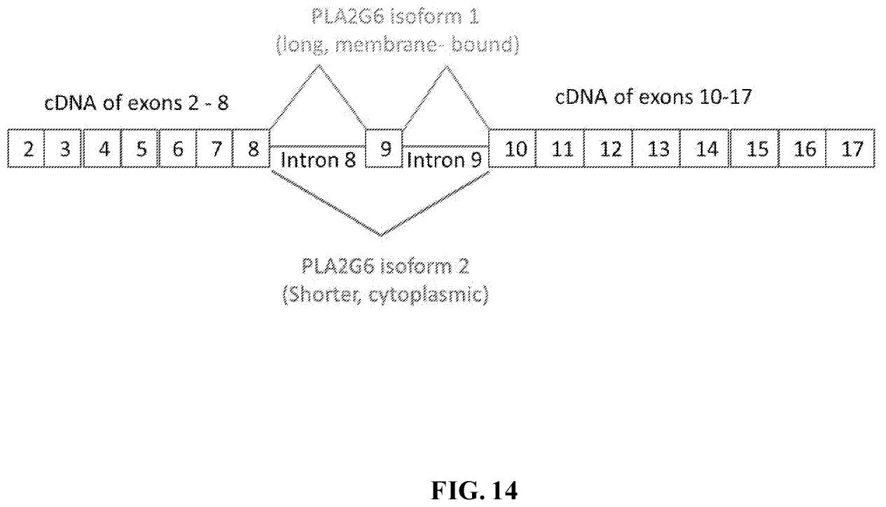

FIG. 14 is a schematic the cDNA/genomic hybrid. The design allows alternate splicing to make a mixture of mRNAs with and without exon 9 thereby encoding both membrane bound and cytoplasmic isoform of the PLA2G6 protein. The cDNA configuration is used for exons 2-8 followed by a switch to the genomic configuration for intron 8, exon 9, and intron 9. This is finally followed by a switch back to cDNA for exons 9-17. This allows the alternative splicing of exon 9 to occur as with the native gene and thereby provides a mixture of cytoplasmic and membrane bound PLA2G6 proteins (isoforms 1 and 2). Exon 1 is not included in the construct because Exon 1 is not needed and does not encode the protein.

A further shortened construct was also produced. Notably, introns do not generally contain critical elements for protein expression. Accordingly, the construct is further developed by deleting the centers of the introns 8 and 9 leaving only the ends of each of introns 8 and 9 where the splicing signals are located.

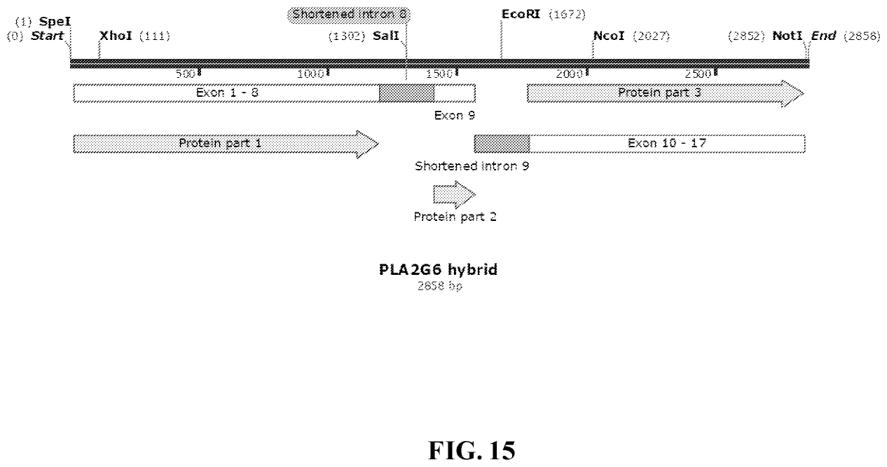

FIG. 15 is a schematic the cDNA/genomic hybrid with shortened intron 8 and intron 9.

SEQ ID NO: 2 is the sequence of an exemplary construct. SEQ ID NO: 2 may be placed into an AAV vector of any capsid serotype with any promoter to allow cell-specific or ubiquitous express at high or low level. The exact deletion of the introns can be larger or smaller as long as the cis elements that allow alternate splicing are retained and the intron still fits within the carrying capacity of AAV vectors.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

Nucleic Acic Sequences
Nucleic acid encoding PLA2G6 isoform 1 (3299 bp)
SEQ ID NO: 1
GTTTGTTTGC GGAAGTAGGA GGAAGTAGAA GTGCTGAGTA AGCCGAGGTG
AGTGACCTCG CGGGTGGGCG GGGCCTGGGG GTCCGTTCCC CAACTTCCTC
GGCGCTCCGG ACTCCCAAGT CTCCGCCGGA CCCTCCTTTG GATATTCCTC
GTGTCTCCGA TTCTGAGACA GAGGGGGAAG ACGGTGGGGC CTCCCCACCT
GCCCCGCAGA AGATGCAGTT CTTTGGCCGC CTGGTCAATA CCTTCAGTGG
CGTCACCAAC TTGTTCTCTA ACCCATTCCG GGTGAAGGAG GTGGCTGTGG
CCGACTACAC CTCGAGTGAC CGAGTTCGGG AGGAAGGGCA GCTGATTCTG
TTCCAGAACA CTCCCAACCG CACCTGGGAC TGCGTCCTGG TCAACCCCAG
GAACTCACAG AGTGGATTCC GACTCTTCCA GCTGGAGTTG GAGGCTGACG
CCCTAGTGAA TTTCCATCAG TATTCTTCCC AGCTGCTACC CTTCTATGAG
AGCTCCCCTC AGGTCCTGCA CACTGAGGTC CTGCAGCACC TGACCGACCT
CATCCGTAAC CACCCCAGCT GGTCAGTGGC CCACCTGGCT GTGGAGCTAG
GGATCCGCGA GTGCTTCCAT CACAGCCGTA TCATCAGCTG TGCCAATTGC
GCGGAGAACG AGGAGGGCTG CACACCCCTG CACCTGGCCT GCCGCAAGGG
TGATGGGGAG ATCCTGGTGG AGCTGGTGCA GTACTGCCAC ACTCAGATGG
ATGTCACCGA CTACAAGGGA GAGACCGTCT TCCATTATGC TGTCCAGGGT
GACAATTCTC AGGTGCTGCA GCTCCTTGGA AGGAACGCAG TGGCTGGCCT
GAACCAGGTG AATAACCAAG GGCTGACCCC GCTGCACCTG GCCTGCCAGC
TGGGGAAGCA GGAGATGGTC CGCGTGCTGC TGCTGTGCAA TGCTCGGTGC
AACATCATGG GCCCCAACGG CTACCCCATC CACTCGGCCA TGAAGTTCTC
TCAGAAGGGG TGTGCGGAGA TGATCATCAG CATGGACAGC AGCCAGATCC
ACAGCAAAGA CCCCCGTTAC GGAGCCAGCC CCCTCCACTG GGCCAAGAAC
GCAGAGATGG CCCGCATGCT GCTGAAACGG GGCTGCAACG TGAACAGCAC
CAGCTCCGCG GGGAACACGG CCCTGCACGT GGCGGTGATG CGCAACCGCT
TCGACTGTGC CATAGTGCTG CTGACCCACG GGGCCAACGC GGATGCCCGC
GGAGAGCACG GCAACACCCC GCTGCACCTG GCCATGTCGA AAGACAACGT
GGAGATGATC AAGGCCCTCA TCGTGTTCGG AGCAGAAGTG GACACCCCGA
ATGACTTTGG GGAGACTCCT ACATTCCTAG CCTCCAAAAT CGGCAGACTT
GTCACCAGGA AGGCGATCTT GACTCTGCTG AGAACCGTGG GGGCCGAATA
CTGCTTCCCA CCCATCCACG GGGTCCCCGC GGAGCAGGGC TCTGCAGCGC
CACATCATCC CTTCTCCCTG GAAAGAGCTC AGCCCCCACC GATCAGCCTA
AACAACCTAG AACTACAGGA TCTCATGCAC ATCTCACGGG CCCGGAAGCC
AGCGTTCATC CTGGGCTCCA TGAGGGACGA GAAGCGGACC CACGACCACC
TGCTGTGCCT GGATGGAGGA GGAGTGAAAG GCCTCATCAT CATCCAGCTC
CTCATCGCCA TCGAGAAGGC CTCGGGTGTG GCCACCAAGG ACCTGTTTGA
CTGGGTGGCG GGCACCAGCA CTGGAGGCAT CCTGGCCCTG GCCATTCTGC
ACAGTAAGTC CATGGCCTAC ATGCGCGGCA TGTACTTTCG CATGAAGGAT
GAGGTGTTCC GGGGCTCCAG GCCCTACGAG TCGGGGCCCC TGGAGGAGTT
CCTGAAGCGG GAGTTTGGGG AGCACACCAA GATGACGGAC GTCAGGAAAC
CCAAGGTGAT GCTGACAGGG ACACTGTCTG ACCGGCAGCC GGCTGAACTC
CACCTCTTCC GGAACTACGA TGCTCCAGAA ACTGTCCGGG AGCCTCGTTT
CAACCAGAAC GTTAACCTCA GGCCTCCAGC TCAGCCCTCA GACCAGCTGG
TGTGGCGGGC GGCCCGAAGC AGCGGGGCAG CTCCTACTTA CTTCCGACCC
AATGGGCGCT TCCTGGACGG TGGGCTGCTG GCCAACAACC CCACGCTGGA
TGCCATGACC GAGATCCATG AGTACAATCA GGACCTGATC CGCAAGGGTC
AGGCCAACAA GGTGAAGAAA CTCTCCATCG TTGTCTCCCT GGGGACAGGG
AGGTCCCCAC AAGTGCCTGT GACCTGTGTG GATGTCTTCC GTCCCAGCAA
CCCCTGGGAG CTGGCCAAGA CTGTTTTTGG GGCCAAGGAA CTGGGCAAGA
TGGTGGTGGA CTGTTGCACG GATCCAGACG GGCGGGCTGT GGACCGGGCA
CGGGCCTGGT GCGAGATGGT CGGCATCCAG TACTTCAGAT TGAACCCCCA
GCTGGGGACG GACATCATGC TGGATGAGGT CAGTGACACA GTGCTGGTCA
ACGCCCTCTG GGAGACCGAG GTCTACATCT ATGAGCACCG CGAGGAGTTC
CAGAAGCTCA TCCAGCTGCT GCTCTCACCC TGAGGGTCCC CAGCCTCTCA
CCGGCCCCAG CTGACCTCGT CCATTCAGCC CCTGCCAGGC CAAGCCCAGC
CACTGCCCTC CCGGGCAGAT CTGGGCCCAG GCACCTCTGA GTCCATAGAC
CAGGCCTGGG AGAATGCCAA GCTGCCTGCC CGAGGCTGGT CCTGAAGGCC
TGTCTCCCAC TAACCCCGCC TTCCAGCACT TTCTGTCATT CCAGGCTGGG
AAAGTCTAGA GCCCCCTTTG GCCCCTTTCC CTGACTGTCA AGGACAACTG
ACTCCCCCAT CAGCTCAAAC ATTAAGGGTA CCCGGGCACA ACCGTACCCC
TGCCCCCAGC CCCAGCCTCC CTGAGGGCCT GCCGGGCTGC CTCTGCCCCA
GCCCCCAGCA AGGGCACTCC CAGGCTTCCT GGTGGGTGCA GCCCACTCCC
TCTGCCCTCT GCTCCGTTCC CTGGGGGCTG GGACTAAAGA AATGGGTGTC
CCCCACCCCA TCAGCTGGGA AAGCCCAGGC CGCAGGAGTG GGATGCCCGT
TGGACTTTGC CCCTCACACT GGCCCAGCCC CTCACACTGC CCCACCCCGA
GAACCCTCAG CTCTCAAAGG TCACTCCTGG GAGTTTCTTC TTCCCAATGG
AAGTGGCTTA AGAGCCAAAA CTGAAATAAA TCATTTGGAT TCAAGTTCA
Nucleic acid encoding PLA2G6 hybrid (2858 bp)
SEQ ID NO: 2
ACTAGTGCCA CCATGCAGTT CTTTGGCCGC CTGGTCAATA CCTTCAGTGG
CGTCACCAAC TTGTTCTCTA ACCCATTCCG GGTGAAGGAG GTGGCTGTGG
CCGACTACAC CTCGAGTGAC CGAGTTCGGG AGGAAGGGCA GCTGATTCTG
TTCCAGAACA CTCCCAACCG CACCTGGGAC TGCGTCCTGG TCAACCCCAG
GAACTCACAG AGTGGATTCC GACTCTTCCA GCTGGAGTTG GAGGCTGACG
CCCTAGTGAA TTTCCATCAG TATTCTTCCC AGCTGCTACC CTTCTATGAG
AGCTCCCCTC AGGTCCTGCA CACTGAGGTC CTGCAGCACC TGACCGACCT
CATCCGTAAC CACCCCAGCT GGTCAGTGGC CCACCTGGCT GTGGAGCTAG
GGATCCGCGA GTGCTTCCAT CACAGCCGTA TCATCAGCTG TGCCAATTGC
GCGGAGAACG AGGAGGGCTG CACACCCCTG CACCTGGCCT GCCGCAAGGG
TGATGGGGAG ATCCTGGTGG AGCTGGTGCA GTACTGCCAC ACTCAGATGG
ATGTCACCGA CTACAAGGGA GAGACCGTCT TCCATTATGC TGTCCAGGGT
GACAATTCTC AGGTGCTGCA GCTCCTTGGA AGGAACGCAG TGGCTGGCCT
GAACCAGGTG AATAACCAAG GGCTGACCCC GCTGCACCTG GCCTGCCAGC
TGGGGAAGCA GGAGATGGTC CGCGTGCTGC TGCTGTGCAA TGCTCGGTGC
AACATCATGG GCCCCAACGG CTACCCCATC CACTCGGCCA TGAAGTTCTC
TCAGAAGGGG TGTGCGGAGA TGATCATCAG CATGGACAGC AGCCAGATCC
ACAGCAAAGA CCCCCGTTAC GGAGCCAGCC CCCTCCACTG GGCCAAGAAC
GCAGAGATGG CCCGCATGCT GCTGAAACGG GGCTGCAACG TGAACAGCAC
CAGCTCCGCG GGGAACACGG CCCTGCACGT GGCGGTGATG CGCAACCGCT
TCGACTGTGC CATAGTGCTG CTGACCCACG GGGCCAACGC GGATGCCCGC
GGAGAGCACG GCAACACCCC GCTGCACCTG GCCATGTCGA AAGACAACGT
GGAGATGATC AAGGCCCTCA TCGTGTTCGG AGCAGAAGTG GACACCCCGA
ATGACTTTGG GGAGACTCCT ACATTCCTAG CCTCCAAAAT CGGCAGACGT
ATGTGCTCTG CACTCTGGGG CTGGAGTGGG GTGAGGGTTG GGTGGATACA
GGGACCGAGG AGGAGGGAAG TCCCAGGCAT CAGGTGCCAG CATCCTGTGT
AGTCGACAGC TGGGACAGGG GACGGAGTGT GGAAAGGAGG GGCCTCTCCT
TTGCATGTTG ACATCATTTC TGATCCTGTG CCATCTCCCC TTCCCCAAAC
CATGACAAGT TGTCACCAGG AAGGCGATCT TGACTCTGCT GAGAACCGTG
GGGGCCGAAT ACTGCTTCCC ACCCATCCAC GGGGTCCCCG CGGAGCAGGG
CTCTGCAGCG CCACATCATC CCTTCTCCCT GGAAAGAGCT CAGCCCCCAC
CGATCAGCCT AAACAACCTA GGTAGGCCTC GCCTCCCGAC TCCCTCTTCT
CCAGCTGGTT CCCAGGCCCC TGGATCACAG CAGGCCCCAC CAAAGCAACA
GGATCCCTAG GACGGAAGTC AGAATTCCCG TGGGCGAGGC ACAGCCCAGG
TTTGATCTAG GGACCTCTGG GGTAGCAGGG CTTGGGGACC CACCTGACCA
CAGCATGCCC TGCTCTGTGC CTCACAGAAC TACAGGATCT CATGCACATC
TCACGGGCCC GGAAGCCAGC GTTCATCCTG GGCTCCATGA GGGACGAGAA
GCGGACCCAC GACCACCTGC TGTGCCTGGA TGGAGGAGGA GTGAAAGGCC
TCATCATCAT CCAGCTCCTC ATCGCCATCG AGAAGGCCTC GGGTGTGGCC
ACCAAGGACC TGTTTGACTG GGTGGCGGGC ACCAGCACTG GAGGCATCCT
GGCCCTGGCC ATTCTGCACA GTAAGTCCAT GGCCTACATG CGCGGCATGT
ACTTTCGCAT GAAGGATGAG GTGTTCCGGG GCTCCAGGCC CTACGAGTCG
GGGCCCCTGG AGGAGTTCCT GAAGCGGGAG TTTGGGGAGC ACACCAAGAT
GACGGACGTC AGGAAACCCA AGGTGATGCT GACAGGGACA CTGTCTGACC
GGCAGCCGGC TGAACTCCAC CTCTTCCGGA ACTACGATGC TCCAGAAACT
GTCCGGGAGC CTCGTTTCAA CCAGAACGTT AACCTCAGGC CTCCAGCTCA
GCCCTCAGAC CAGCTGGTGT GGCGGGCGGC CCGAAGCAGC GGGGCAGCTC
CTACTTACTT CCGACCCAAT GGGCGCTTCC TGGACGGTGG GCTGCTGGCC
AACAACCCCA CGCTGGATGC CATGACCGAG ATCCATGAGT ACAATCAGGA
CCTGATCCGC AAGGGTCAGG CCAACAAGGT GAAGAAACTC TCCATCGTTG
TCTCCCTGGG GACAGGGAGG TCCCCACAAG TGCCTGTGAC CTGTGTGGAT
GTCTTCCGTC CCAGCAACCC CTGGGAGCTG GCCAAGACTG TTTTTGGGGC
CAAGGAACTG GGCAAGATGG TGGTGGACTG TTGCACGGAT CCAGACGGGC
GGGCTGTGGA CCGGGCACGG GCCTGGTGCG AGATGGTCGG CATCCAGTAC
TTCAGATTGA ACCCCCAGCT GGGGACGGAC ATCATGCTGG ATGAGGTCAG
TGACACAGTG CTGGTCAACG CCCTCTGGGA GACCGAGGTC TACATCTATG
AGCACCGCGA GGAGTTCCAG AAGCTCATCC AGCTGCTGCT CTCACCCTGA
GCGGCCGC

### REFERENCES

Adams, D., M. Midei, J. Dastgir, C. Flora, R. J. Molinari, F. Heerinckx, S. Endemann, P. Atwal, P. Milner and M. S. Shchepinov (2020). “Treatment of infantile neuroaxonal dystrophy with RT001: A di-deuterated ethyl ester of linoleic acid: Report of two cases.” JIMD Rep 54 (1): 54-60.
Altuame, F. D., G. Foskett, P. S. Atwal, S. Endemann, M. Midei, P. Milner, M. A. Salih, M. Hamad, M. Al-Muhaizea, M. Hashem and F. S. Alkuraya (2020). “The natural history of infantile neuroaxonal dystrophy.” Orphanet J Rare Dis 15.
Atwal, P. S., M. Midei, D. Adams, A. Fay, F. Heerinckx and P. Milner (2020). “The infantile neuroaxonal dystrophy rating scale (INAD-RS).” Orphanet J Rare Dis 15.
Bailey, R. M., D. Armao, S. Nagabhushan Kalburgi and S. J. Gray (2018). “Development of Intrathecal AAV9 Gene Therapy for Giant Axonal Neuropathy.” Mol Ther Methods Clin Dev 9:160-171.
Bao, S., D. J. Miller, Z. Ma, M. Wohltmann, G. Eng, S. Ramanadham, K. Moley and J. Turk (2004). “Male mice that do not express group VIA phospholipase A2 produce spermatozoa with impaired motility and have greatly reduced fertility.” J Biol Chem 279 (37): 38194-38200.
Beharier, O., V. A. Tyurin, J. P. Goff, J. Guerrero-Santoro, K. Kajiwara, T. Chu, Y. Y. Tyurina, C. M. St Croix, C. T. Wallace, S. Parry, W. T. Parks, V. E. Kagan and Y. Sadovsky (2020). “PLA2G6 guards placental trophoblasts against ferroptotic injury.” Proc Natl Acad Sci USA 117 (44): 27319-27328.
Dalwadi, D. A., L. Torrens, J. Abril-Fornaguera, R. Pinyol, C. Willoughby, J. Posey, J. M. Llovet, C. Lanciault, D. W. Russell, M. Grompe and W. E. Naugler (2021). “Liver Injury Increases the Incidence of HCC following AAV Gene Therapy in Mice.” Mol Ther 29 (2): 680-690.
Engel, L. A., Z. Jing, D. E. O'Brien, M. Sun and P. T. Kotzbauer (2010). “Catalytic function of PLA2G6 is impaired by mutations associated with infantile neuroaxonal dystrophy but not dystonia-parkinsonism.” PLoS One 5 (9): e12897.
Farina, L., N. Nardocci, M. G. Bruzzone, L. D'Incerti, G. Zorzi, L. Verga, M. Morbin and M. Savoiardo (1999). “Infantile neuroaxonal dystrophy: neuroradiological studies in 11 patients.” Neuroradiology 41 (5): 376-380.
Forsell, P. K., A. O. Olsson, E. Andersson, L. Nallan and M. H. Gelb (2005). “Polychlorinated biphenyls induce arachidonic acid release in human platelets in a tamoxifen sensitive manner via activation of group IVA cytosolic phospholipase A2-alpha.” Biochem Pharmacol 71 (1-2): 144-155.
Gitiaux, C., A. Kaminska, N. Boddaert, G. Barcia, S. Guéden, S. N. The Tich, P. De Lonlay, S. Quijano-Roy, M. Hully, Y. Péréon and I. Desguerre (2018). “PLA2G6-associated neurodegeneration: Lessons from neurophysiological findings.” Eur J Paediatr Neurol 22 (5): 854-861.
Glanzman, A. M., E. Mazzone, M. Main, M. Pelliccioni, J. Wood, K. J. Swoboda, C. Scott, M. Pane, S. Messina, E. Bertini, E. Mercuri and R. S. Finkel (2010). “The Children's Hospital of Philadelphia Infant Test of Neuromuscular Disorders (CHOP INTEND): test development and reliability.” Neuromuscul Disord 20 (3): 155-161.
Gong, Y., A. Berenson, F. Laheji, G. Gao, D. Wang, C. Ng, A. Volak, R. Kok, V. Kreouzis, I. M. Dijkstra, S. Kemp, C. A. Maguire and F. Eichler (2019). “Intrathecal Adeno-Associated Viral Vector-Mediated Gene Delivery for Adrenomyeloneuropathy.” Hum Gene Ther 30 (5): 544-555.
Gray, S. J., S. Nagabhushan Kalburgi, T. J. McCown and R. Jude Samulski (2013). “Global CNS gene delivery and evasion of anti-AAV-neutralizing antibodies by intrathecal AAV administration in non-human primates.” Gene Ther 20 (4): 450-459.
Gregory, A. and S. Hayflick (1993). Neurodegeneration with Brain Iron Accumulation Disorders Overview. GeneReviews®. M. P. Adam, H. H. Ardinger, R. A. Pagon et al. Seattle (WA), University of Washington, Seattle
Copyright © 1993-2022, University of Washington, Seattle. GeneReviews is a registered trademark of the University of Washington, Seattle. All rights reserved.
Gregory, A. and S. J. Hayflick (2005). “Neurodegeneration with brain iron accumulation.” Folia Neuropathol 43 (4): 286-296.
Hayflick, S. J., M. A. Kurian and P. Hogarth (2018). “Neurodegeneration with brain iron accumulation.” Handb Clin Neurol 147:293-305.
Hinderer, C., P. Bell, N. Katz, C. H. Vite, J. P. Louboutin, E. Bote, H. Yu, Y. Zhu, M. L. Casal, J. Bagel, P. O'Donnell, P. Wang, M. E. Haskins, T. Goode and J. M. Wilson (2018). “Evaluation of Intrathecal Routes of Administration for Adeno-Associated Viral Vectors in Large Animals.” Hum Gene Ther 29 (1): 15-24.
Hinderer, C., N. Katz, J. P. Louboutin, P. Bell, H. Yu, M. Nayal, K. Kozarsky, W. T. O'Brien, T. Goode and J. M. Wilson (2016). “Delivery of an Adeno-Associated Virus Vector into Cerebrospinal Fluid Attenuates Central Nervous System Disease in Mucopolysaccharidosis Type II Mice.” Hum Gene Ther 27 (11): 906-915.
Hordeaux, J., E. L. Buza, C. Dyer, T. Goode, T. W. Mitchell, L. Richman, N. Denton, C. Hinderer, N. Katz, R. Schmid, R. Miller, G. R. Choudhury, M. Horiuchi, K. Nambiar, H. Yan, M. Li and J. M. Wilson (2020). “Adeno-Associated Virus-Induced Dorsal Root Ganglion Pathology.” Hum Gene Ther 31 (15-16): 808-818.
Hordeaux, J., C. Hinderer, E. L. Buza, J. P. Louboutin, T. Jahan, P. Bell, J. A. Chichester, A. F. Tarantal and J. M. Wilson (2019). “Safe and Sustained Expression of Human Iduronidase After Intrathecal Administration of Adeno-Associated Virus Serotype 9 in Infant Rhesus Monkeys.” Hum Gene Ther 30 (8): 957-966.
Hordeaux, J., C. Hinderer, T. Goode, N. Katz, E. L. Buza, P. Bell, R. Calcedo, L. K. Richman and J. M. Wilson (2018). “Toxicology Study of Intra-Cisterna Magna Adeno-Associated Virus 9 Expressing Human Alpha-L-Iduronidase in Rhesus Macaques.” Mol Ther Methods Clin Dev 10:79-88.
Iodice, A., C. Spagnoli, G. G. Salerno, D. Frattini, G. Bertani, P. Bergonzini, F. Pisani and C. Fusco (2017). “Infantile neuroaxonal dystrophy and PLA2G6-associated neurodegeneration: An update for the diagnosis.” Brain Dev 39 (2): 93-100.
Kinghorn, K. J., J. I. Castillo-Quan, F. Bartolome, P. R. Angelova, L. Li, S. Pope, H. M. Cochemé, S. Khan, S. Asghari, K. P. Bhatia, J. Hardy, A. Y. Abramov and L. Partridge (2015). “Loss of PLA2G6 leads to elevated mitochondrial lipid peroxidation and mitochondrial dysfunction.” Brain 138 (Pt 7): 1801-1816.
Kingsman, S. M., K. Mitrophanous and J. C. Olsen (2005). “Potential oncogene activity of the woodchuck hepatitis post-transcriptional regulatory element (WPRE).” Gene Ther 12 (1): 3-4.
Kurokawa, Y., H. Osaka, T. Kouga, E. Jimbo, K. Muramatsu, S. Nakamura, Y. Takayanagi, T. Onaka, S. I. Muramatsu and T. Yamagata (2021). “Gene Therapy in a Mouse Model of Niemann-Pick Disease Type C1.” Hum Gene Ther 32 (11-12): 589-598.
Li, D., C. Liu, C. Yang, D. Wang, D. Wu, Y. Qi, Q. Su, G. Gao, Z. Xu and Y. Guo (2017). “Slow Intrathecal Injection of rAAVrh10 Enhances its Transduction of Spinal Cord and Therapeutic Efficacy in a Mutant SOD1 Model of ALS.” Neuroscience 365:192-205.
Lin, G., P. T. Lee, K. Chen, D. Mao, K. L. Tan, Z. Zuo, W. W. Lin, L. Wang and H. J. Bellen (2018). “Phospholipase PLA2G6, a Parkinsonism-Associated Gene, Affects Vps26 and Vps35, Retromer Function, and Ceramide Levels, Similar to α-Synuclein Gain.” Cell Metab 28 (4): 605-618.e606.
Marshall, M. S., Y. Issa, B. Jakubauskas, M. Stoskute, V. Elackattu, J. N. Marshall, W. Bogue, D. Nguyen, Z. Hauck, E. Rue, S. Karumuthil-Melethil, V. Zaric, M. Bosland, R. B. van Breemen, M. I. Givogri, S. J. Gray, S. J. Crocker and E. R. Bongarzone (2018). “Long-Term Improvement of Neurological Signs and Metabolic Dysfunction in a Mouse Model of Krabbe's Disease after Global Gene Therapy.” Mol Ther 26 (3): 874-889.
Mascalchi, M., F. Mari, B. Berti, E. Bartolini, M. Lenge, A. Bianchi, L. Antonucci, F. M. Santorelli, B. Garavaglia and R. Guerrini (2017). “Fast Progression of Cerebellar Atrophy in PLA2G6-Associated Infantile Neuronal Axonal Dystrophy.” Cerebellum 16 (3): 742-745.
Mathiesen, S. N., J. L. Lock, L. Schoderboeck, W. C. Abraham and S. M. Hughes (2020). “CNS Transduction Benefits of AAV-PHP.eB over AAV9 Are Dependent on Administration Route and Mouse Strain.” Mol Ther Methods Clin Dev 19:447-458.
Meadows, A. S., F. J. Duncan, M. Camboni, K. Waligura, C. Montgomery, K. Zaraspe, B. J. Naughton, W. G. Bremer, C. Shilling, C. M. Walker, B. Bolon, K. M. Flanigan, K. L. McBride, D. M. McCarty and H. Fu (2015). “A GLP-Compliant Toxicology and Biodistribution Study: Systemic Delivery of an rAAV9 Vector for the Treatment of Mucopolysaccharidosis IIIB.” Hum Gene Ther Clin Dev 26 (4): 228-242.
Meyer, E., M. A. Kurian and S. J. Hayflick (2015). “Neurodegeneration with Brain Iron Accumulation: Genetic Diversity and Pathophysiological Mechanisms.” Annu Rev Genomics Hum Genet 16:257-279.
Meyer, K., L. Ferraiuolo, L. Schmelzer, L. Braun, V. McGovern, S. Likhite, O. Michels, A. Govoni, J. Fitzgerald, P. Morales, K. D. Foust, J. R. Mendell, A. H. Burghes and B. K. Kaspar (2015). “Improving single injection CSF delivery of AAV9-mediated gene therapy for SMA: a dose-response study in mice and nonhuman primates.” Mol Ther 23 (3): 477-487.
Nathwani, A. C., E. G. Tuddenham, S. Rangarajan, C. Rosales, J. McIntosh, D. C. Linch, P. Chowdary, A. Riddell, A. J. Pie, C. Harrington, J. O'Beirne, K. Smith, J. Pasi, B. Glader, P. Rustagi, C. Y. Ng, M. A. Kay, J. Zhou, Y. Spence, C. L. Morton, J. Allay, J. Coleman, S. Sleep, J. M. Cunningham, D. Srivastava, E. Basner-Tschakarjan, F. Mingozzi, K. A. High, J. T. Gray, U. M. Reiss, A. W. Nienhuis and A. M. Davidoff (2011). “Adenovirus-associated virus vector-mediated gene transfer in hemophilia B.” N Engl J Med 365 (25): 2357-2365.
Piguet, F., T. de Saint Denis, E. Audouard, K. Beccaria, A. André, G. Wurtz, R. Schatz, S. Alves, C. Sevin, M. Zerah and N. Cartier (2021). “The Challenge of Gene Therapy for Neurological Diseases: Strategies and Tools to Achieve Efficient Delivery to the Central Nervous System.” Hum Gene Ther 32 (7-8): 349-374.
Rosenberg, J. B., M. G. Kaplitt, B. P. De, A. Chen, T. Flagiello, C. Salami, E. Pey, L. Zhao, R. J. Ricart Arbona, S. Monette, J. P. Dyke, D. J. Ballon, S. M. Kaminsky, D. Sondhi, G. A. Petsko, S. M. Paul and R. G. Crystal (2018). “AAVrh.10-Mediated APOE2 Central Nervous System Gene Therapy for APOE4-Associated Alzheimer's Disease.” Hum Gene Ther Clin Dev 29 (1): 24-47.
Ruehl-Fehlert, C., B. Kittel, G. Morawietz, P. Deslex, C. Keenan, C. R. Mahrt, T. Nolte, M. Robinson, B. P. Stuart and U. Deschl (2003). “Revised guides for organ sampling and trimming in rats and mice—part 1.” Exp Toxicol Pathol 55 (2-3): 91-106.
Strokin, M., K. L. Seburn, G. A. Cox, K. A. Martens and G. Reiser (2012). “Severe disturbance in the Ca2+ signaling in astrocytes from mouse models of human infantile neuroaxonal dystrophy with mutated Pla2g6.” Hum Mol Genet 21 (12): 2807-2814.
Wada, H., S. Kojo and K. Seino (2013). “Mouse models of human INAD by Pla2g6 deficiency.” Histol Histopathol 28 (8): 965-969.
Wada, H., T. Yasuda, I. Miura, K. Watabe, C. Sawa, H. Kamijuku, S. Kojo, M. Taniguchi, I. Nishino, S. Wakana, H. Yoshida and K. Seino (2009). “Establishment of an improved mouse model for infantile neuroaxonal dystrophy that shows early disease onset and bears a point mutation in Pla2g6.” Am J Pathol 175 (6): 2257-2263.
Whaler, S. (2018). Novel therapeutic strategies in NBIA: A gene therapy approach for PLA2G6-associated neurodegeneration. PhD, University College London.

1. A viral vector comprising:
a viral capsid; and
an expression cassette comprising, in order:
a SYN1 promoter or an EF1a promoter;
a nucleic acid sequence encoding a PLA2G6 gene; and
a poly(A) signal,
wherein the expression cassette does not comprise a woodchuck hepatitis virus post-transcriptional regulatory element between the nucleic acid sequence encoding a PLA2G6 gene and the poly(A) signal.

2. The vector of claim 1, wherein the nucleic acid sequence encoding the PLA2G6 gene encodes isoform 1 of PLA2G6 gene and/or isoform 2 of the of PLA2G6 gene.

3. The vector of claim 1, wherein the expression cassette does not comprise a woodchuck hepatitis virus post-transcriptional regulatory element.

4. The vector of claim 1, wherein the viral capsid is an AAV9 capsid.

5. The vector of claim 4, further comprising inverted terminal repeats (ITRs) flanking the expression cassette.

6. The vector of claim 5, wherein the ITRs are AAV2 ITRs.

7. The vector of claim 1, wherein the vector enables expression of the expression cassette in non-neuronal tissue.

8. The vector of claim 7, wherein the vector enables expression of the expression cassette in muscular tissue.

9. The vector of claim 7, wherein the promoter is a SYN1 promoter.

10. The vector of claim 7, wherein the promoter is a EF1a promoter.

11. A method of treating Infantile Neuroaxonal Dystrophy (INAD) in a subject, the method comprising administering to the subject a composition comprising:
a viral capsid; and
an expression cassette comprising, in order:
a SYN1 and/or EF1a promoter;
a nucleic acid sequence encoding a PLA2G6 gene; and
a poly(A) signal,
wherein the expression cassette does not comprise a woodchuck hepatitis virus post-transcriptional regulatory element between the nucleic acid sequence encoding a PLA2G6 gene and the poly(A) signal.

12. The vector of claim 11, wherein the nucleic acid sequence encoding the PLA2G6 gene encodes isoform 1 of PLA2G6 gene and/or isoform 2 of the of PLA2G6 gene.

13. The vector of claim 11, wherein the expression cassette does not comprise a woodchuck hepatitis virus post-transcriptional regulatory element.

14. The vector of claim 11, wherein the viral capsid is an AAV9 capsid.

15. The vector of claim 14, further comprising inverted terminal repeats (ITRs) flanking the expression cassette.

16. The vector of claim 15, wherein the ITRs are AAV2 ITRs.

17. The vector of claim 11, wherein the vector enables expression of the expression cassette in non-neuronal tissue.

18. The vector of claim 17, wherein the vector enables expression of the expression cassette in muscular tissue.

19. The vector of claim 17, wherein the promoter is a SYN1 promoter.

20. The vector of claim 17, wherein the promoter is a EF1a promoter.

21. A nucleic acid encoding the PLA2G6 gene, comprising a nucleic acid encoding portions of both isoforms of the PLA2G6 gene.

22. The nucleic acid of claim 21, wherein the nucleic acid comprises hybrid cDNA and genomic DNA.

23. The nucleic acid of claim 22, wherein the nucleic acid allows for alternate splicing to result in a mixture of mRNAs with and without exon 9.

24. The nucleic acid of claim 22, wherein the nucleic acid comprises cDNA for exons 2-8 of the PLA2G6 gene.

25. The nucleic acid of claim 24, wherein the nucleic acid comprises genomic DNA for intron 8, exon 9, and intron 9 of the PLA2G6 gene.

26. The nucleic acid of claim 25, wherein the nucleic acid comprises cDNA for exons 9-17 of the PLA2G6 gene.

27. The nucleic acid of claim 26, wherein the nucleic acid does not comprise exon 1 of the PLA2G6 gene.

28. The nucleic acid of claim 26, wherein portions of the center of introns 8 and 9 of the PLA2G6 gene are removed without removing the ends of introns 8 and 9 of the PLA2G6 gene.

29. The nucleic acid of claim 21, wherein the nucleic acid comprises the nucleic acid sequence of SEQ ID NO: 2.

30. The nucleic acid of claim 1, wherein the nucleic acid is packaged into an AAV vector.


SEQUENCE LISTING (2 sequences)

SEQ ID NO: 1 - DNA, 3299 bp, synthetic construct
  source 1..3299: mol_type = other DNA
gtttgtttgcggaagtaggaggaagtagaagtgctgagtaagccgaggtgagtgacctcgcgggtgggcggggcctgggggtccgttccccaacttcctcggcgctccggactcccaagtctccgccggaccctcctttggatattcctcgtgtctccgattctgagacagagggggaagacggtggggcctccccacctgccccgcagaagatgcagttctttggccgcctggtcaataccttcagtggcgtcaccaacttgttctctaacccattccgggtgaaggaggtggctgtggccgactacacctcgagtgaccgagttcgggaggaagggcagctgattctgttccagaacactcccaaccgcacctgggactgcgtcctggtcaaccccaggaactcacagagtggattccgactcttccagctggagttggaggctgacgccctagtgaatttccatcagtattcttcccagctgctacccttctatgagagctcccctcaggtcctgcacactgaggtcctgcagcacctgaccgacctcatccgtaaccaccccagctggtcagtggcccacctggctgtggagctagggatccgcgagtgcttccatcacagccgtatcatcagctgtgccaattgcgcggagaacgaggagggctgcacacccctgcacctggcctgccgcaagggtgatggggagatcctggtggagctggtgcagtactgccacactcagatggatgtcaccgactacaagggagagaccgtcttccattatgctgtccagggtgacaattctcaggtgctgcagctccttggaaggaacgcagtggctggcctgaaccaggtgaataaccaagggctgaccccgctgcacctggcctgccagctggggaagcaggagatggtccgcgtgctgctgctgtgcaatgctcggtgcaacatcatgggccccaacggctaccccatccactcggccatgaagttctctcagaaggggtgtgcggagatgatcatcagcatggacagcagccagatccacagcaaagacccccgttacggagccagccccctccactgggccaagaacgcagagatggcccgcatgctgctgaaacggggctgcaacgtgaacagcaccagctccgcggggaacacggccctgcacgtggcggtgatgcgcaaccgcttcgactgtgccatagtgctgctgacccacggggccaacgcggatgcccgcggagagcacggcaacaccccgctgcacctggccatgtcgaaagacaacgtggagatgatcaaggccctcatcgtgttcggagcagaagtggacaccccgaatgactttggggagactcctacattcctagcctccaaaatcggcagacttgtcaccaggaaggcgatcttgactctgctgagaaccgtgggggccgaatactgcttcccacccatccacggggtccccgcggagcagggctctgcagcgccacatcatcccttctccctggaaagagctcagcccccaccgatcagcctaaacaacctagaactacaggatctcatgcacatctcacgggcccggaagccagcgttcatcctgggctccatgagggacgagaagcggacccacgaccacctgctgtgcctggatggaggaggagtgaaaggcctcatcatcatccagctcctcatcgccatcgagaaggcctcgggtgtggccaccaaggacctgtttgactgggtggcgggcaccagcactggaggcatcctggccctggccattctgcacagtaagtccatggcctacatgcgcggcatgtactttcgcatgaaggatgaggtgttccggggctccaggccctacgagtcggggcccctggaggagttcctgaagcgggagtttggggagcacaccaagatgacggacgtcaggaaacccaaggtgatgctgacagggacactgtctgaccggcagccggctgaactccacctcttccggaactacgatgctccagaaactgtccgggagcctcgtttcaaccagaacgttaacctcaggcctccagctcagccctcagaccagctggtgtggcgggcggcccgaagcagcggggcagctcctacttacttccgacccaatgggcgcttcctggacggtgggctgctggccaacaaccccacgctggatgccatgaccgagatccatgagtacaatcaggacctgatccgcaagggtcaggccaacaaggtgaagaaactctccatcgttgtctccctggggacagggaggtccccacaagtgcctgtgacctgtgtggatgtcttccgtcccagcaacccctgggagctggccaagactgtttttggggccaaggaactgggcaagatggtggtggactgttgcacggatccagacgggcgggctgtggaccgggcacgggcctggtgcgagatggtcggcatccagtacttcagattgaacccccagctggggacggacatcatgctggatgaggtcagtgacacagtgctggtcaacgccctctgggagaccgaggtctacatctatgagcaccgcgaggagttccagaagctcatccagctgctgctctcaccctgagggtccccagcctctcaccggccccagctgacctcgtccattcagcccctgccaggccaagcccagccactgccctcccgggcagatctgggcccaggcacctctgagtccatagaccaggcctgggagaatgccaagctgcctgcccgaggctggtcctgaaggcctgtctcccactaaccccgccttccagcactttctgtcattccaggctgggaaagtctagagccccctttggcccctttccctgactgtcaaggacaactgactcccccatcagctcaaacattaagggtacccgggcacaaccgtacccctgcccccagccccagcctccctgagggcctgccgggctgcctctgccccagcccccagcaagggcactcccaggcttcctggtgggtgcagcccactccctctgccctctgctccgttccctgggggctgggactaaagaaatgggtgtcccccaccccatcagctgggaaagcccaggccgcaggagtgggatgcccgttggactttgcccctcacactggcccagcccctcacactgccccaccccgagaaccctcagctctcaaaggtcactcctgggagtttcttcttcccaatggaagtggcttaagagccaaaactgaaataaatcatttggattcaagttca

SEQ ID NO: 2 - DNA, 2858 bp, synthetic construct
  source 1..2858: mol_type = other DNA
actagtgccaccatgcagttctttggccgcctggtcaataccttcagtggcgtcaccaacttgttctctaacccattccgggtgaaggaggtggctgtggccgactacacctcgagtgaccgagttcgggaggaagggcagctgattctgttccagaacactcccaaccgcacctgggactgcgtcctggtcaaccccaggaactcacagagtggattccgactcttccagctggagttggaggctgacgccctagtgaatttccatcagtattcttcccagctgctacccttctatgagagctcccctcaggtcctgcacactgaggtcctgcagcacctgaccgacctcatccgtaaccaccccagctggtcagtggcccacctggctgtggagctagggatccgcgagtgcttccatcacagccgtatcatcagctgtgccaattgcgcggagaacgaggagggctgcacacccctgcacctggcctgccgcaagggtgatggggagatcctggtggagctggtgcagtactgccacactcagatggatgtcaccgactacaagggagagaccgtcttccattatgctgtccagggtgacaattctcaggtgctgcagctccttggaaggaacgcagtggctggcctgaaccaggtgaataaccaagggctgaccccgctgcacctggcctgccagctggggaagcaggagatggtccgcgtgctgctgctgtgcaatgctcggtgcaacatcatgggccccaacggctaccccatccactcggccatgaagttctctcagaaggggtgtgcggagatgatcatcagcatggacagcagccagatccacagcaaagacccccgttacggagccagccccctccactgggccaagaacgcagagatggcccgcatgctgctgaaacggggctgcaacgtgaacagcaccagctccgcggggaacacggccctgcacgtggcggtgatgcgcaaccgcttcgactgtgccatagtgctgctgacccacggggccaacgcggatgcccgcggagagcacggcaacaccccgctgcacctggccatgtcgaaagacaacgtggagatgatcaaggccctcatcgtgttcggagcagaagtggacaccccgaatgactttggggagactcctacattcctagcctccaaaatcggcagacgtatgtgctctgcactctggggctggagtggggtgagggttgggtggatacagggaccgaggaggagggaagtcccaggcatcaggtgccagcatcctgtgtagtcgacagctgggacaggggacggagtgtggaaaggaggggcctctcctttgcatgttgacatcatttctgatcctgtgccatctccccttccccaaaccatgacaagttgtcaccaggaaggcgatcttgactctgctgagaaccgtgggggccgaatactgcttcccacccatccacggggtccccgcggagcagggctctgcagcgccacatcatcccttctccctggaaagagctcagcccccaccgatcagcctaaacaacctaggtaggcctcgcctcccgactccctcttctccagctggttcccaggcccctggatcacagcaggccccaccaaagcaacaggatccctaggacggaagtcagaattcccgtgggcgaggcacagcccaggtttgatctagggacctctggggtagcagggcttggggacccacctgaccacagcatgccctgctctgtgcctcacagaactacaggatctcatgcacatctcacgggcccggaagccagcgttcatcctgggctccatgagggacgagaagcggacccacgaccacctgctgtgcctggatggaggaggagtgaaaggcctcatcatcatccagctcctcatcgccatcgagaaggcctcgggtgtggccaccaaggacctgtttgactgggtggcgggcaccagcactggaggcatcctggccctggccattctgcacagtaagtccatggcctacatgcgcggcatgtactttcgcatgaaggatgaggtgttccggggctccaggccctacgagtcggggcccctggaggagttcctgaagcgggagtttggggagcacaccaagatgacggacgtcaggaaacccaaggtgatgctgacagggacactgtctgaccggcagccggctgaactccacctcttccggaactacgatgctccagaaactgtccgggagcctcgtttcaaccagaacgttaacctcaggcctccagctcagccctcagaccagctggtgtggcgggcggcccgaagcagcggggcagctcctacttacttccgacccaatgggcgcttcctggacggtgggctgctggccaacaaccccacgctggatgccatgaccgagatccatgagtacaatcaggacctgatccgcaagggtcaggccaacaaggtgaagaaactctccatcgttgtctccctggggacagggaggtccccacaagtgcctgtgacctgtgtggatgtcttccgtcccagcaacccctgggagctggccaagactgtttttggggccaaggaactgggcaagatggtggtggactgttgcacggatccagacgggcgggctgtggaccgggcacgggcctggtgcgagatggtcggcatccagtacttcagattgaacccccagctggggacggacatcatgctggatgaggtcagtgacacagtgctggtcaacgccctctgggagaccgaggtctacatctatgagcaccgcgaggagttccagaagctcatccagctgctgctctcaccctgagcggccgc